(19) United States
(12) Patent Application Publication
(10) Pub. No.: US 2006/0210647 A1
(43) Pub. Date: Sep. 21, 2006

(54) Copper-dependent non-traditional pro-inflammatory cytokine export and methods, compositions and kits relating thereto

(75) Inventors: Thomas Maciag, Portland, ME (US); Lorraine A. Maciag, South Freeport, ME (US); Anna Mandinova, Ipswich, MA (US); Lazar Mandinov, Ipswich, MA (US); Igor Prudovsky, Cape Elizabeth, ME (US); Stephen Bellum, South Portland, ME (US); Raffaella Soldi, Windham, ME (US); Cinzia Bagala, Rome (IT)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Maine Medical Center Research Institute

(21) Appl. No.: 11/439,397

(22) Filed: May 22, 2006

Related U.S. Application Data
(62) Division of application No. 10/786,223, filed on Feb. 23, 2004.

(30) Foreign Application Priority Data
Aug. 26, 2002 (WO) PCT/US02/27247

(51) Int. Cl. A61K 33/26; A61K 38/17

(52) U.S. Cl. 424/646; 514/12

(57) ABSTRACT

The present invention relates to the discovery that non-traditional export of certain pro-inflammatory cytokines lacking a signal sequence from a cell can be inhibited by copper chelation and/or administration to the cell of a truncated form of S100A13 lacking the basic residue portion. Further, copper chelation inhibits, inter alia, neointima formation, macrophage infiltration and associated inflammation, cell proliferation, secretion of extracellular matrix, intimal thickening, adventitial angiogenesis, restenosis, and the like, associated with vascular vessel injury. Thus, the present invention provides novel methods of preventing and treating, and for identifying novel compounds also useful as therapeutics for, such conditions.

### BACKGROUND OF THE INVENTION

The prototype members of the interleukin 1 (IL1) and fibroblast growth factor (FGF) gene families are well recognized for their receptor-dependent inflammatory and angiogenic activities in vitro and in vivo (Dinarello, 1994, FASEB J. 8:1314-1325; Krakauer, 1986, Crit. Rev. Immunol. 6:213-244; Dinarello, 1998, Int. Rev. Immunol. 16:457-499; Maini and Taylor, 2000, Annu. Rev. Med. 51:207-229; Blum and Miller, 2001, Annu. Rev. Med. 52:15-27; Burgess and Maciag, 1989, Annu. Rev. Med. 58:575-606; Friesel and Maciag, 1999, Thromb. Haemost. 82:748-754; McKeehan et al., 1998, Prog. Nucleic Acid Res. Mol. Biol. 59:135-176; Vlodavsky et al. 1996, Cancer Metastasis Rev. 15:177-186), yet these prototypes lack a signal peptide sequence to direct their export through the classical secretion pathway mediated by the endoplasmic reticulum-Golgi apparatus (Jaye et al., 1986, Science 233:541-545; Abraham et al., 1986, Science 233:545-548; Lomedico et al., 1984, Nature 312:458-462). Interestingly, crystallographic studies have demonstrated that the prototype members of the IL1 and FGF gene families exhibit a high level of structural homology (Carter et al., 1988, Proteins 3:121-129; Zhang et al., 1991, Proc. Natl. Acad. Sci. USA 88:3446-3450; Zhu et al., 1991, Science 251:90-93; Eriksson et al., 1991, Proc. Natl. Acad. Sci. USA 88:3441-3445) despite their unremarkable sequence similarities (Thomas et al., 1985, Proc. Natl. Acad. Sci. USA 82:6409-6413). While the FGF gene family evolved only three genes lacking a signal peptide sequence (Burgess and Maciag, 1989, Annu. Rev. Med. 58:575-606; Friesel and Maciag, 1999, Thromb. Haemost. 82:748-754; McKeehan et al., 1998, Prog. Nucleic Acid Res. Mol. Biol. 59:135-176), eight of the ten members of the IL1 gene family lack this structural feature (Smith, et al. 2000; Kumar, et al. 2000). Thus, it is important to understand and define the non-classical pathways utilized by these signal peptide-less cytokines for export since this information may ultimately prove to be valuable for the clinical management of inflammatory and angiogenic-dependent events.

The release of the FGF1 and IL1α prototypes is regulated by convergent yet distinct pathways which utilize cellular stress to mediate export of these polypeptides into the extracellular compartment (Tarantini et al., 2001, J. Biol. Chem. 276:5147-5151; Tarantini et al., 1995, J. Biol. Chem. 270:29039-29042). It is known that FGF1 is released in response to stress as a latent homodimer which requires intracellular oxidation of a conserved cysteine residue at position 30 (Tarantini et al., 1995). This event enables FGF1 to interact with the extravesicular p40 domain of synaptotagmin (Syt)1 and S100A13 (Tarantini et al., 1998, J. Biol. Chem. 273:22209-22216; LaVallee et al., 1998, J. Biol. Chem. 273:22217-22223; Carreira et al., 1998, J. Biol. Chem. 273:22224-22231; Landriscina et al., 2001, J. Biol. Chem. 276:22544-22552), and these interactions facilitate the release of FGF1 as a multiprotein aggregate containing p40 Syt1 and S100A13 (Landriscina et al., 2001). Interestingly, while temperature stress induces the release of the mature but not the precursor form of IL1α, the expression of precursor IL1α represses the release of FGF1 in response to stress (Tarantini et al., 2001).

The oxidative stress required for the formation of the Cys30 FGF1 homodimer does not involve the induction of a classical stress-induced transcriptional response. Rather, the ability of Syt1 and S100A13 to associate with Cu²⁺ is utilized to regulate the formation of this multiprotein export complex in response to stress (Tarantini et al., 1998; LaVallee et al., 1998; Carreira et al., 1998; Landriscina et al., 2001). Further, because (i) FGF1, S100A13 and Syt1 are Cu²⁺-binding proteins (Shing, 1988, J. Biol. Chem. 263:9059-9062; Landriscina et al., 2001; Engleka and Maciag, 1992, J. Biol. Chem. 267:11307-11315), (ii) Cu²⁺-induced oxidation facilitates the self assembly of a FGF1, p40 Syt1 and S100A13 complex in a cell-free system (Landriscina et al., 2001), (iii) S100A 13 expression facilitates the release of FGF1 independent of transcription (Landriscina et al., 2001), and (iv) the Cu²⁺ chelator, tetrathiomolybdate (TTM) inhibits the release of FGF1 in response to stress (Landriscina, et al. 2001), it is likely that intracellular Cu²⁺ metabolism plays a role in the stress-induced oxidative event which facilitates the release of FGF1. However, despite the importance of IL-1 in various processes and conditions, the mechanism of its release was poorly understood. Further, the role of copper in the non-traditional release of IL-1, if any, was also not understood. Thus, there is a long term need for the understanding of the mechanism for the release of IL-1 from a cell, as well as a need for therapeutics for inhibiting such release in order to treat or prevent conditions mediated by the release of this cytokine from a cell. The present invention meets these needs.

In addition, restenosis after percutaneous coronary interventions occurs in 10 to 50% of patients, and remains the Achilles' heel of interventional cardiology (Libby et al. 1997, N. Engl. J. Med. 337:418-419, Serruys et al. 1991, N. Engl. J. Med. 324:13-17, Serruys et al. 1994, N. Engl. J. Med. 331:489-495, Erbel et al. 1998, N. Engl. J. Med. 339:1672-1678, Kastrati et al. 2001, Am. J. Cardiol. 87:34-39 and Serruys et al. 2001, N. Engl. J. Med. 344:1117-1124). Although in-stent restenosis is quite distinct from restenosis after balloon angioplasty, which involves additionally vessel elastic recoil as well as negative vessel remodeling and vasoconstriction, there is also a common essential pathobiological process in both of them, histologically comprised largely of neointimal formation (Moreno et al., 1999, Am. J. Cardiol. 84:462-466, Mach, 2000, Arterioscier. Thromb. Vasc. Biol. 20:1699-1700, Lafont et al., 1995, Circ. Res. 76:996-1002; and Andersen et al., 1996, Circulation 93:1716-1724).

The neointima development is a natural response of the arterial wall to injury, and is based on time-dependent infiltration of the arterial wall with inflammatory cells as well as on up-regulation of growth factors and inflammatory cytokines (Wang et al., 2000, Biochem. Biophys. Res. Commun. 271:138-143 and Ward et al., 1997, Arterioscler. Thromb. Vasc. Biol. 17:2461-2470). This leads to migration of vascular smooth muscle cells (SMC) from the vessel media to the intima where they continue to proliferate and deposit extracellular matrix (Bendeck et al., 1994, Circ. Res. 75:539-545, Fishel et al., 1995, J. Clin. Invest. 95:377-387 and Wempe et al. 1997, Arterioscler. Thromb. Vasc. Biol. 17:2471-2478).

IL1 and FGF1, the prototype members of the IL1 and FGF gene families are well recognized for their receptor-dependent inflammatory and mitogenic activities in vitro and in vivo (Burgess et al., 1989, Annu. Rev. Biochem. 58:575-606; Friesel et al., 1999, Thromb. Haemost. 82:748-754, Dinarello et al., 1988, FASEB J. 2:108-115, Dinarello et al., 1994, FASEB J. 8:1314-1325 and Maini et al., 2000, Annu. Rev. Med. 51:207-229). FGF1, which has become recognized as a key mediator of angiogenesis, is also an important regulator of a range of cellular behaviors including migration, proliferation, differentiation, and survival. Since FGF1 is also a powerful mitogen for coronary smooth muscle cells, it contributes considerably to the pathogenesis of restenosis after coronary interventions (Law et al., 1996, J. Clin. Invest. 98:1897-1905). Additionally, IL1 as anextracellular protein may be significant to restenosis due to its multiple roles as both a proinflammatory cytokine and as a regulator of endothelial cell behavior (Hancock et al. 1994, Am J Pathol., 145:1008-1014). It is through IL1 function as an inflammatory agent that it can recruit macrophages, which are the richest cellular source of FGF1 in the body, to sites of inflammation and/or physiological stress. Indeed, there is a direct correlation between the infiltration of macrophages population and neointima formation after balloon injury (Moreno et al., 1996, Circulation 94:3098-3102 and Pietersma et al., 1995, Circulation., 91:1320-1325).

The release of FGF1 and IL1 is regulated by convergent yet distinct pathways, which utilize stress to mediate export of these polypeptides into the extracellular compartment (Tarantini et al., 1995, J. Biol. Chem. 270:29039-29042 and Tarantini et al., 2001, J. Biol. Chem. 276:5147-5151). It is known that FGF1 is released in response to stress as a biologically inactive homodimer, which is formed through a disulfide linkage between the conserved cysteine residues at position 30 (Tarantini et al., 1995, J. Biol. Chem. 270:29039-29042, Tarantini et al., 2001, J. Biol. Chem. 276:5147-515 and Engleka et al., 1992, J. Biol. Chem. 267:11307-11315). This event enables FGF1 to interact with a small calcium binding protein S100A13 and the extravesicular p40 domain of synaptotagmin 1 (Syt1), making FGF dimer a component of a larger non-covalently associated multiprotein complex containing p40 Syt1 and S100A13 (Landriscina et al., 2001, J. Biol. Chem. 276:25549-25557; LaVallee et al., 1998, J. Biol. Chem. 273:22217-22223; and Landriscina et al., 2001, J. Biol. Chem. 276:22544-22552). Like FGF1, IL1 is a signal peptide-less protein whose release is stimulated by stress conditions such as injury, inflammation and hypoxia.

Despite previous studies suggesting the important role of FGF1 and IL-1 in negative remodeling and restenosis, and the increased mortality and morbidity related to these processes in treatment of vascular disease, there is a long-felt need to understand the mechanism for the release of these leader-less proteins from a cell and for the development of therapeutics for treatment and prophylaxis of restenosis in a mammal. The present invention meets these needs.

### SUMMARY OF THE INVENTION

The invention includes a method of inhibiting interleukin-1 alpha (IL-1) release from a cell. The method comprises administering an effective amount of an IL-1 release inhibitor to said cell, thereby inhibiting IL-1 release from said cell.

In one aspect, the release is stress-induced, and further wherein the IL-1 release inhibitor is selected from the group consisting of a copper chelator and a S100A13, or a fragment thereof.

In another aspect, the S100A13 fragment is the truncated protein S100A13 BR.

In yet another aspect, the copper chelator is tretrathiomolybdate (TTM).

The invention includes a method of treating a condition mediated by stress-induced release of IL-1 from a cell. The method comprises administering an effective amount of a copper chelator to said cell, thereby treating said condition.

The invention includes a method of inhibiting neointima formation following vessel injury in a mammal. The method comprises administering to the mammal an IL-1 release inhibiting amount of a copperchelator, thereby inhibiting the neointima formation.

The invention includes a method of inhibiting macrophage infiltration following vessel injury in a mammal. The method comprises administering to the mammal an effective amount of a copper chelator, thereby inhibiting the macrophage infiltration.

In one aspect, the macrophage infiltration is associated with inflammation.

The invention includes a method of inhibiting cell proliferation associated with arterial wall injury. The method comprises administering an effective amount of a copper chelator to the mammal, thereby inhibiting the cell proliferation.

In one aspect, the cell is a vascular smooth muscle cell and further wherein the copper chelator is TTM and the injury is caused by balloon angioplasty.

The invention includes a method of inhibiting secretion of extracellular matrix following arterial wall injury in a mammal. The method comprises inhibiting non-traditional export of at least one of FGF-1 and IL-1 from a cell at the site of the injury, and further wherein the export is inhibited by administering an effective amount of a copper chelator to the mammal, thereby inhibiting the secretion of extracellular matrix in the mammal.

The invention includes a method of inhibiting neointimal thickening associated with arterial wall injury in a mammal. The method comprises inhibiting non-traditional export of at least one of FGF-1 and IL-1 from a cell at the site of the injury, and further wherein the export is inhibited by administering an effective amount of a copper chelator to the mammal, thereby inhibiting the neointimal thickening in the mammal.

The invention includes a method of inhibiting adventitial angiogenesis associated with arterial wall injury in a mammal. The method comprises inhibiting non-traditional export of at least one of FGF-1 and IL-1 from a cell at the site of the injury, and further wherein the export is inhibited by administering an effective amount of a copper chelator to the mammal, thereby inhibiting the adventitial angiogenesis in the mammal.

The invention includes a method of identifying a compound useful for inhibiting adventitial angiogenesis associated with arterial wall injury in a mammal. The method comprises contacting a cell with a compound and comparing the level of release of a leader-less pro-inflammatory cytokine by the cell in response to temperature stress with the level of release of the cytokine from an otherwise identical cell not contacted with the compound in response to the temperature stress, wherein a decrease in the level of release of the leader-less pro-inflammatory cytokine by the contacted with the compound with the level of release of the cytokine from the otherwise identical cell not contacted with the compound is an indication that the compound inhibits the angiogenesis, thereby identifying a compound useful for inhibiting adventitial angiogenesis associated with arterial wall injury in a mammal.

In one aspect, the leader-less pro-inflammatory cytokine is selected from the group consisting of FGF-1 and IL-1.

In another aspect, the invention includes a compound identified by this method.

The invention includes a kit for inhibiting relase of IL-1 from a cell. The kit comprises an effective amount of an IL-1 release inhibitor, the kit further comprising an applicator and an instructional material for the use thereof.

The invention includes a kit for treating a condition mediated by stress-induced release of IL-1 from a cell. The kit comprises an effective amount of a copper chelator, the kit further comprising an applicator and an instructional material for the use thereof.

The invention also includes a kit for inhibiting neointima formation following vessel injury in a mammal. The kit comprises an IL-1 release inhibiting amount of a copper chelator, the kit further comprising an applicator and an instructional material for the use thereof.

The invention includes a kit for inhibiting restenosis following vessel injury in a mammal. The kit comprises an effective amount of a copper chelator, the kit further comprising an applicator and an instructional material for the use thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A and 1B, is an image of a gel depicting that IL1α binds immobilized Cu²⁺. FIG. 1A is an image depicting an IL1α immunoblot analysis of recombinant human IL1α (1 μg) resolved using Cu²⁺-chelator affinity chromatography (Hi Trap Chelation; Amersham Pharmacia Biotech) as a function of the concentration of imidazole as indicated by “mM Imidazole”. The flow through (“flow”) and 50 mM EDTA (“EDTA”) elution fractions are also shown. FIG. 1B is an image depicting conditioned medium obtained from heat-shocked IL1α (mature form) NIH 3T3 cell transfectants resolved by Cu²⁺-chelator affinity chromatography as subjected to IL1α immunoblot analysis as described in Panel A.

FIG. 2, comprising FIGS. 2A and 2B, is an image depicting the Cu²⁺-dependent interaction of IL1α with the carboxy-terminus of S100A13. FIG. 2A is an image depicting the interaction of recombinant human IL1α with S100A13, which was assessed by the incubation of these proteins in PBS followed by ultracentrifugation and S100A13-immunoblot analysis of the pellet fractions. The data demonstrate that S100A13 was not present in the pellet fraction when incubated in the presence of Cu²⁺ but in the absence of IL1α. FIG. 2B is an image depicting the interaction of S100A13 and IL1α, which was further analyzed by the incubation of the recombinant protein in 100% (NH₄)₂SO₄ as described elsewhere herein.

FIG. 3, comprising FIGS. 3A and 3B, is an image depicting that S100A13 is involved in the release of IL1α. FIG. 3A is an image demonstrating the effect of myc-S100A13 expression on IL1α release in response to heat shock. Briefly, myc-S100A13 and mature (m) IL1α-βGal, myc-S100A13 and precursor (p) IL1α-βGal, insert-less vector, and pIL1α-βGal insert-less vector and mIL1α-βGal NIH 3T3 cell cotransfectants were subjected to heat shock. Conditioned media were collected and processed as described by LaVallee, et al. (1998). Immunoprecipitated and eluted proteins were resolved by 8% and 12% acrylamide SDS-PAGE, respectively, and evaluated by IL1α (Upper Panel) and Myc (Lower Panel) immunoblot analysis. FIG. 3B is an image depicting the ability of myc-S100A13 and mIL1α to associate with heparin. Briefly, myc-S100A13 and mIL1α-βGal NIH 3T3 cell cotransfectants and insert-less vector and mIL1α-βGal NIH 3T3 cell cotransfectants were subjected to heat shock (2 hours at 42 C). Conditioned media were collected, subjected to 100% (NH₄)₂SO₄ fractionation, centrifuged and analyzed by heparin-Sepharose-affinity (LaVallee, et al. 1998). The eluted proteins were resolved by 10% acrylamide SDS-PAGE and evaluated by IL1α (Upper Panel) and Myc (Lower Panel) immunoblot analysis.

FIG. 4, comprising FIGS. 4A and 4B, is an image depicting deletion of the basic residue-rich carboxy-terminus (about nine amino acid residues of S100A13) mediates the mutant to function as a dominant negative effector of IL1α release. FIG. 4A is an image depicting that the recombinant form of S100A13 lacking the basic residue-rich (BR) domain of S100A13 (termed “S100A13ABR”) was incubated with recombinant IL1α as described in FIG. 2A at the molar ratios indicated. FIG. 4B is an image depicting that the deletion mutant, S100A13ΔBR and IL1α NIH 3T3 cell cotransfectants were subjected to heat shock and, following DTT treatment, conditioned media were concentrated and immunoprecipitated with anti-IL1α antibody for the evaluation of IL1α release. Immunoprecipitated proteins were resolved by 12% (w/v) SDS-PAGE, respectively, and evaluated using IL1α immunoblot analysis.

FIG. 5, comprising FIGS. 5A and 5B, depict the involvement of Cu²⁺ in IL1α release. FIG. 5A is an image depicting results obtained using NIH 3T3 cells stably transfected with mIL1α-βGal. The cells were incubated for 18 hours at 37 C in the absence and presence of the Cu²⁺ chelator, tetrathiomolybdate (TTM) as indicated and the untreated and treated cells either maintained at 37 C or subjected to heat shock as described in FIG. 3. The conditioned medium was evaluated for IL1α-βGal release by IL1α immunoblot analysis, and cell lysates from TTM-treated cells were used to monitor the intracellular level of IL1α-βGal expression. The TTM-negative control cell lysate exhibited a similar level of IL1α-βGal expression. FIG. 5B is an image depicting release of IL1α-βGal in myc-S100A13 and IL1α-βGal NIH 3T3 cell cotransfectants and in insert-less vector and mIL1α-βGal NIH 3T3 cell transfectants in the presence and absence actinomycin D (10 μg/ml), as indicated, in response to heat shock. Conditioned media were processed and evaluated for IL1α-βGal immunoblot analysis as described in Tarantini et al. (2001).

FIG. 6, comprising FIGS. 6A through 6F, depicting neointimal formation 14 days after balloon injury. FIGS. 6A-6D are images depicting representative photomicrographs of cross sections of carotid arteries stained with hematoxilin-eosin (magnification ×10). FIG. 6A is an image depicting a representative cross section of the control group not treated with TTM. FIG. 6B is an image depicting the effects of TTM administration for 2 weeks before and 2 weeks after the injury. FIG. 6C is an image depicting the effects of TTM administration 1 week before and 2 weeks after the injury. FIG. 6D is an image depicting the effects of TTM administration for 2 weeks after the injury but not before. FIG. 6E is a bar graph showing intima/media (I/M) ratio (mean+SEM) in all 4 groups of rats. The data depicted demonstrate that each TTM regimen led to significant decrease in I/M ratio when compared to the controls. Moreover, the best results occurred in the group treated with TTM 2 weeks before and 2 weeks after the injury. FIG. 6F is a linear graph showing the ceruloplasmin level in all 4 groups before and after injury.

FIG. 7, comprising FIGS. 7A and 7B, depicts regression analysis. The data demonstrate that I/M ratio depends on ceruloplasmin level at the day of the injury as depicted in FIG. 7A, as well as on the change in serum ceruloplasmin after TTM treatment as depicted in FIG. 7B.

FIG. 8, comprising FIGS. 8A and 8B, depicts the effects of TTM administration. FIG. 8A is a bar graph showing intima/media ratio (mean+SEM) in 5 groups of rats, which were treated with TTM for 2 weeks before the injury and than the TTM was withheld either on the day of the injury or 4, 6, 8 and 10 days after the balloon injury. The best results regarding inhibition of neointimal formation estimated by the I/M ratio occur in the group treated with TTM 2 weeks before and more than 6 days after the injury. FIG. 8B depicts a linear graph showing the ceruloplasmin level in all 5 groups before and after injury.

FIG. 9, comprising FIGS. 9A-9J, is an image depicting the effect of TTM after arterial balloon injury. FIGS. 9A-9D are images depicting photomicrographs of rat carotid artery at 4 days after arterial balloon injury. FIGS. 9F-9I depict images of photomicrographs of rat carotid artery at 7 days after arterial balloon injury. Hematoxylin-eosin staining demonstrated no difference in the neointima development between the controls (FIG. 9A) and TTM-treated rats (FIG. 9B) 4 days after the injury. However, by day 7 after the injury the neointimal development becomes more pronounced in the controls (FIG. 9F) than in the TTM-treated group (FIG. 9G). FIGS. 9C and 9D and FIGS. 9H and 9I are images depicting as follows: FIG. 9C depicts CD11b (MAC1) immunostaining (magnification ×20); FIG. 9D depicts artery of TIM-free rat 4 days after the injury (control); FIG. 9D depicts artery of TTM-treated rat 4 days after the injury (control). FIG. 9H depicts artery of TTM-free rat 7 days after the injury (control); and FIG. 9I depicts artery of TTM-treated rat 7 days after the injury (control). The data demonstrate a marked increase of MAC1 positive cells 4 and 7 days after the injury in the controls as compared to the TIM-treated rats.

Bar graphs showing the CD11b-positive cells counted in the neointima 4 days (FIG. 9E) and 7 days (FIG. 9J) after the injury in the controls and the TTM-treated rats.

FIG. 10, comprising FIGS. 10A-10H), are images depicting photomicrographs demonstrating the effects of TTM. Photomicrographs of rat carotid artery 4 days (FIGS. 10A and 10B), 7 days (FIGS. 10D and 10E) and 14 days (FIGS. 10G and 10H) after arterial balloon injury. FIGS. 10A, 10B, 10D and 10E depict slides that were immunolabeled with an anti-SM α-actin antibody whereas FIGS. 10G and 10H are images depicting slides that were immunolabeled with PCNA. For all images, the magnification was ×20. FIGS. 10A, 10D and 10E are images depicting TTM free animals whereas FIGS. 10B, 10E, and 10H represent carotid arteries obtained from TTM-treated rats. FIG. 10C depicts a bar graph showing αSMA cells counted in the neointima at 4 days, and FIG. 10F depicts αSMA cells counted in the neointima 7 days after the injury in the controls and the TTM-treated rats. FIG. 10J depicts a bar graph demonstrating the difference in PCNA positive cells counted 14 days after the injury in the controls and TTM treated rats.

FIG. 11, comprising FIGS. 11A-11H), is an image depicting photomicrographs of rat carotid artery 14 days after arterial balloon injury. FIGS. 11A and 11B are images depicting slides that were immunolabeled using an anti-S100A13 antibody (magnification ×20). FIGS. 11C and 11D are images depicting slides that were immunolabeled using anti IL1α antibody. FIGS. 11E and 11F are images depicting slides that were labeled with anti p40 antibody. FIGS. 11G and 11H are images depicting slides that were immunolabeled with anti phosphatidylserine antibody. The data demonstrate a difference between positive cells 14 days after the injury in the controls (FIGS. 11A, 11C, 11D, and 11G) as compared with the TTM-treated rats (FIGS. 11B, 11D, 11F, and 11H).

### DETAILED DESCRIPTION OF THE INVENTION

Definitions:

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

By the term “applicator” as the term is used herein, is meant any device including, but not limited to, a hypodermic syringe, a pipette, an intravenous infusion, topical cream and the like, for administering a molecule or compound (e.g., a IL-1 release inhibitor such as, but not limited to, a chemical compound, an antibody, nucleic acid, protein) and/or composition of the invention to a mammal.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment” as applied to a nucleic acid, may ordinarily be at least about 20 nucleotides in length, typically, at least about 50 nucleotides, more typically, from about 50 to about 200 nucleotides, preferably, at least about 200 to about 300 nucleotides, even more preferably, at least about 300 nucleotides to about 400 nucleotides, yet even more preferably, at least about 400 to about 500, even more preferably, at least about 500 nucleotides to about 600 nucleotides, yet even more preferably, at least about 600 to about 700, even more preferably, at least about 700 nucleotides to about 800 nucleotides, yet even more preferably, at least about 800 to about 900, even more preferably, at least about 900 nucleotides to about 1000 nucleotides, yet even more preferably, at least about 1000 to about 1100, even more preferably, at least about 1100 nucleotides to about 1200 nucleotides, yet even more preferably, at least about 1200 to about 1300, even more preferably, at least about 1300 nucleotides to about 1400 nucleotides, yet even more preferably, at least about 1400 to about 1500, at least about 1500 to about 1550, even more preferably, at least about 1550 nucleotides to about 1600 nucleotides, yet even more preferably, at least about 1600 to about 1620 and most preferably, the nucleic acid fragment will be greater than about 1625 nucleotides in length.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 75% homology.

As used herein, “inhibiting IL-1 release from a cell,” as used herein, means mediating any detectable decrease in the level of IL-1 outside a cell, such as the level of IL-1 detectable in tissue culture media obtained from the in vitro culture of the cell, or any decrease in the level of IL-1 detected in a fluid derived from or in contact with a cell in vivo or in vitro.

The term “IL-1 release inhibitor,” includes, but is not limited to, any substance or compound that mediates a detectable decrease in the level of IL-1 released from a cell compared with the level of IL-1 released from the same cell prior to administration of a compound to the cell or compared with the level of IL-1 released from an otherwise identical cell to which the compound is not administered.

As used herein, an “instructional material” includes a publication, a recording, a diagram, or any other medium of expression, which can be used to communicate the usefulness of the nucleic acid, peptide, and/or composition of the invention in the kit for effecting alleviation of the various diseases or disorders recited herein. Optionally, or alternately, the instructional material may describe one or more methods of alleviation the diseases or disorders in a cell or a tissue of a mammal and/or for identifying a useful compound. The instructional material of the kit of the invention may, for example, be affixed to a container, which contains the nucleic acid, peptide, chemical compound and/or composition of the invention or be shipped together with a container, which contains the nucleic acid, peptide, chemical composition, and/or composition. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids, which have been substantially purified from other components, which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized, upon the other. By way of example, a promoter operably linked to the coding region of a gene is able to promote transcription of the coding region.

Preferably, when the nucleic acid encoding the desired protein further comprises a promoter/regulatory sequence, the promoter/regulatory sequence is positioned at the 5′ end of the desired protein coding sequence such that it drives expression of the desired protein in a cell. Together, the nucleic acid encoding the desired protein and its promoter/regulatory sequence comprise a “transgene.”

“Constitutive” expression is a state in which a gene product is produced in a living cell under most or all physiological conditions of the cell.

“Inducible” expression is a state in which a gene product is produced in a living cell in response to the presence of a signal in the cell.

A “recombinant polypeptide” is one, which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

As used herein, the term “transgenic mammal” means a mammal, the germ cells of which, comprise an exogenous nucleic acid.

As used herein, to “treat” means reducing the frequency with which symptoms of a disease or condition are experienced by a mammal, or altering the natural history and/or progression of the disease in a mammal.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, N.Y.; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “portion” of a polynucleotide means at least at least about fifteen to about fifty sequential nucleotide residues of the polynucleotide. It is understood that a portion of a polynucleotide may include every nucleotide residue of the polynucleotide.

By the term “specifically binds,” as used herein, is meant an antibody which recognizes and binds a chitinase-like molecule, but does not substantially recognize or bind other molecules in a sample.

A “prophylactic” treatment is a treatment administered to a subject who does not exhibit signs of a disease or exhibits only early signs of the disease for the purpose of decreasing the risk of developing pathology associated with the disease.

“Preventing” a disease, as the term is used herein, means that the onset of the disease is delayed, and/or that the symptoms of the disease will be decreased in intensity and/or frequency, when a chitinase-like molecule is administered compared with the onset and/or symptoms in the absence of the inhibitor.

A “therapeutic” treatment is a treatment administered to a subject who exhibits signs of pathology for the purpose of diminishing or eliminating those signs.

Description

The present invention provides a novel method for inhibiting the non-traditional export of the leader-less peptide, IL-1, from a cell in response to stress. The method is based, inter alia, on the discovery that IL-1, which lacks a leader sequence, is released from a cell in response to stress via formation of a high molecular weight protein complex. Further, formation of the complex is mediated by and/or requires copper and interaction of IL-1 with S100A13. Moreover, the present invention relates to administration of a copper chelator to inhibit release of IL-1. In addition, the invention provides a novel method of inhibiting release of IL-1 using at least a portion of S100A13, preferably, a truncated form of S100A13 lacking the basic residue portion of the full-length molecule.

The invention provides a novel method of inhibiting the non-traditional release of a pro-inflammatory cytokine, e.g., IL-1 and FGF1, from a cell using a copper chelator. Additionally, the present invention relates to inhibition of, among other things, restenosis, macrophage infiltration, neointima formation, neointimal thickening, cell proliferation, deposition of extracellular matrix, and the like, following injury to a blood vessel. This is because, as more fully set forth elsewhere herein, the data disclosed herein demonstrate that inhibition of release of, e.g., IL-1 and/or FGF1, using a copper chelating compound inhibited a cell mediated response, including restenosis, macrophage infiltration, neointima formation, cell proliferation, deposition of extracellular matrix, and the like. Inhibition of such processes treats or prevents various conditions associated therewith, and the invention therefore provides novel therapeutics useful for treating or preventing conditions mediated by non-traditional export of cytokines from cells in response to cell stress and injury.

A. Method of Inhibiting Non-Traditional Protein Export from a Cell

The invention provides a novel method of inhibiting interleukin-1 alpha (IL-1) release from a cell. The method comprises administering an IL-1 release inhibitor thereby preventing non-traditional export of IL-1 from the cell. Such an inhibitor includes, but is not limited to, a copper chelator, an S100A13 molecule, or a fragment thereof. This is because the data disclosed herein demonstrate, for the first time, that non-traditional export of IL-1, which lacks a leader sequence and is not released from a cell via traditional ER-Golgi protein release mechanism, requires formation of a high molecular weight protein complex and that export of IL-1 is inhibited by administering a copper chelator or by administering a truncated version of S100A13 lacking the basic residue domain of the protein.

The skilled artisan, based upon the disclosure provided herein, would understand that although the chelator used to demonstrate inhibition of IL-1 export was TTM, that the present invention is in no way limited to use of this particular copper chelator. Rather, one skilled in the art would appreciate that the invention encompasses any copper chelator that reduces the level of bioavailable copper in a cell. That is, a copper chelator binds copper such that the metal cannot participate in cellular processes. The skilled artisan, armed with the teachings of this invention, would readily appreciate that the copper chelator encompasses a wide plethora of compounds well-known in the art, and such compounds as are developed in the future, that inhibit copper participation in biological processes in a cell. Thus, TTM is only an exemplar of such a copper chelator, but the invention is not limited to this, or any other, copper chelator.

Similarly, the skilled artisan would understand, based upon the disclosure provided herein, that the invention is not limited to the particular fragment of S100A13 (i.e., S100A13 BR) to inhibit IL-1 export from a cell. That is, one of ordinary skill in the art would appreciate, armed with the teachings provided herein, that various fragments of S100A13 can be used to inhibit the interaction of S100A13 with IL-1, such that export of IL-1 is inhibited. This is because the disclosure provided herein provides methods for determining whether a fragment or variant of S100A13 inhibits export of IL-1, and the skilled artisan would be able to identify and isolate fragments and variants of S100A13 exhibiting the desired characteristic of inhibiting export of IL-1 compared with the level of IL-1 export from a cell in response to stress in the absence of the S100A13 fragment or variant being assessed. Such experimentation would not be undue to one skilled in the art, since the art routinely screens such protein variants and fragments for those having such a desired characteristic. Thus, using the assays set forth herein, or such as assays as are known in the art to detect export of a protein from a cell, the routineer would be able to isolate additional fragments or variants of S100A13 that inhibit export of IL-1 from a cell in response to stress. Therefore, the present invention encompasses administering S100A13 BR, either as a protein or as a nucleic acid encoding the protein, and various fragments thereof as could be readily identified by one skilled in the art based upon the disclosure provided herein.

Further, although the invention demonstrates that the release of IL-1 in response to stress can be selectively inhibited where the stress is exposure of the cell to increased temperature, the present invention is not limited to this type of stress. That is, the skilled artisan, based upon the disclosure provided herein, would appreciate that the present invention encompasses inhibiting the release of IL-1 in response to various cellular stressors, including, but not limited to, heat mediated by stress (e.g., heat shock). Instead, one skilled in the art would understand that the invention encompasses methods for inhibiting release of IL-1 from a cell as a result of a wide variety of stresses, including, but not limited to, heat.

The invention further encompasses a method of treating a condition mediated by stress-induced release of IL-1 from a cell. The method comprises administering an effective amount of a copper chelator the cell. This is because, as discussed previously elsewhere herein, it has been discovered that decreasing the level of bioavailable copper, by, for instance, chelating copper using, among other things, TTM, inhibits release of IL-1 from a cell. Therefore, the skilled artisan would understand that where a condition is mediated by IL-1 release from a cell, such condition can be treated by administering a copper chelator since such treatment inhibits IL-1 export from a cell.

The skilled artisan would understand that the identity and/or amount of the chelator administered can be readily determined according to well-established criteria known in the pharmaceutical arts. Similarly, the route of administration and dosing regimen can also be readily determined by one skilled in the art based upon the disclosure provided herein. For instance, the data disclosed herein demonstrate that the level of plasma ceruloplasmin can serve as an indicator of the level of copper and can also be used to assess the effectiveness of the copper chelating therapy thereby determining the dose, route of administration, and the like. Additionally, the effective dose of the inhibitor can be assessed by determining the level of IL-1 release from a cell before, during and after the treatment, thereby assessing an effective level of the IL-1 release inhibitor.

Without wishing to be limited to any particular dose or treatment regimen, the data disclosed herein demonstrate that the skilled artisan can, once armed with the teachings of the present invention, determine the dose and treatment regimen as exemplified herein using an art-recognized animal model of human restenosis. Thus, once armed with the teachings provided herein, one skilled in the art can determine, as disclosed herein, the dose, route of administration, the dosing regimen, and the like, for each copper chelator used especially in light of various parameters well-known in the pharmacological arts. Such parameters include, but are not limited to, the condition being treated, and the age, weight and condition of the mammal being treated, and these, and other factors, are well-known to one skilled in the art. Therefore, the skilled artisan could readily determine the dose and regiment for each condition that is being treated by inhibiting IL-1 release from a cell.

B. Methods of Treating and Preventing

The present invention encompasses a method of inhibiting neointima formation following vessel injury in a mammal. The method comprises administering to a mammal, an IL-1 release inhibiting amount of a copperchelator. This is because, as discussed previously elsewhere herein, administering a copper chelator inhibits IL-1 release from a cell, such that administering a copper chelator to a mammal treats or prevents a disease mediated by export of IL-1 release from a cell. Such disease includes, but is not limited to, neointima formation following vessel injury. Without wishing to be bound by any particular theory, the data disclosed herein demonstrate that in an art-recognized model of human restenosis following vessel damage mediated by balloon angioplasty, administration of the copper chelator, TTM, inhibited neointima formation. Thus, the skilled artisan, armed with the teachings of the present invention, would understand that the invention includes a method of inhibiting neointima formation in a mammal by administering a copper chelator to the mammal.

As pointed out previously elsewhere herein, the data disclosed herein demonstrate that formation of a high molecular weight protein complex, and subsequent release of a pro-inflammatory cytokine (e.g., IL-1 and FGF1), requires copper and interaction of IL-1 with S100A13. More specifically, the data demonstrate, for the first time, that chelation of copper using, e.g., the powerful chelator TTM, inhibited release of IL-1 from a cell in response to stress.

The skilled artisan would also appreciate, based upon the disclosure provided herein and as discussed previously elsewhere herein, that the present invention encompasses use of a wide variety of copper chelating compounds to inhibit the non-traditional export of IL-1 and FGF1 from a cell. That is, once armed with the teachings of the invention, one skilled in the art would understand that the invention includes use of compounds other than TTM that decrease the level of copper available in a cell, including such compounds that may be developed in the future. The skilled artisan would understand that a copper chelator could inhibit IL-1 and FGF1 release from a cell as demonstrated, for the first time, elsewhere herein. Armed with these teachings, one skilled in the art would understand the invention is in no way limited to use of TTM, or any other copper chelator in particular; instead, the skilled artisan would understand the invention includes use of a wide plethora of copper-chelating compounds, including, but not limited to, TTM.

Further, the skilled artisan would understand that the amount of the chelator administered can be readily determined according to well-established criteria known in the pharmaceutical arts. Similarly, the route of administration and dosing regimen can also be readily determined by one skilled in the art based upon the disclosure provided herein. For instance, the data disclosed herein demonstrate that the level of plasma ceruloplasmin can serve as an indicator of the level of bioavailable copper (e.g., copper that is available to participate in cellular processes) and can also be used to determine the dose, route of administration, and the like, to assess the effectiveness of the copper chelating therapy.

Without wishing to be limited to any particular dose or treatment regimen, the data disclosed herein demonstrate that the skilled artisan could, once armed with the teachings of the present invention, determine the dose and treatment regimen as exemplified herein using an art-recognized animal model of human restenosis. That is, the data disclosed herein demonstrate the successful inhibition of neointima formation in a rat model of vessel damage relating to balloon angioplasty, by administration of various amounts of the copper chelator, TIM, and various dosage and treatment regimens. Further, the data disclosed herein demonstrate that the copper chelator can be administered orally, by simply including the compound in the drinking water. However, the skilled artisan would appreciate that the invention is not limited to any particular dose or route of administration; rather, the compound can be administered via a wide variety of routes and administration dosages and regimens, and the invention encompasses them as well.

Thus, once armed with the teachings provided herein, one skilled in the art could determine, as disclosed herein, to adjust the dose, route of administration, the dosing regimen, and the like, for each copper chelator used and according to various parameters well-known in the pharmacological arts. Such parameters include, but are not limited to, the condition being treated, and the age, weight and condition of the mammal being treated.

The skilled artisan, armed with the teachings of the present invention, would understand that the dose and treatment regimen can be readily determined for each mammal treated, as exemplified herein using a rat model of restenosis, using methods well known in the relevant art. That is, one skilled in the art would appreciate that the level of neointima formed can be determined as, for example, disclosed elsewhere herein, by comparing the level of, inter alia, serum ceruloplamin activity, I/M ratio, infiltration by macrophages, deposition of extracellular matrix, cell proliferation, and the like, in an animal to which the chelator is administered with the level in an otherwise identical animal to which the chelator is not provided. The skilled artisan would understand, based upon the disclosure provided herein, that the amount of chelator can be readily adjusted and the therapeutic effects thereof can be monitored during the course of treatment and the optimal parameters can be determined for each mammal treated.

It will be appreciated by one of skill in the art, when armed with the present disclosure including the methods detailed herein, that the invention is not limited to inhibition of neointima formation once restenosis has occurred. Particularly, restenosis need not occur and the chelator can be administered prophylactically to prevent neointima formation. That is, the data disclosed herein demonstrate that a copper chelator administered prior to and after vessel injury can prevent neointima formation such that neointima formation, and any restenosis or deleterious effect thereof, can be prevented such that the methods of the invention can actually prevent restenosis, not just treat it once it has occurred.

One of skill in the art, when armed with the disclosure herein, would appreciate that inhibiting the release of IL-1 and/or FGF1 can be used to prevent a diasease or condition mediated by the release of such pro-inflammatory cytokines. Such disease or condition includes, but is not limited to, neointima formation, restenosis, macrophage infiltration, cell proliferation, increase in intima/media ratio, and the like. Given these etiologies and the methods disclosed elsewhere herein, the skilled artisan can recognize and prevent an inflammatory disease in a mammal wherein the disease relates to a pro-inflammatory response that can be inhibited by administration of a copper chelator. This is because the data disclosed herein demonstrate that administration of a copper chelator, including, but not limited to, TTM, prevented restenosis in a mammal, as well as other responses (i.e., macrophage infiltration, increase in the intima/media, deposition of extracellular matrix, and cell proliferation). Accordingly, the skilled artisan would appreciate, based on the disclosure provided elsewhere herein, that the present invention includes a method of preventing disease in a mammal and comprising administering a copper chelator.

The invention encompasses administration of a IL1-inhibitor to practice the methods of the invention; the skilled artisan would understand, based on the disclosure provided herein, how to formulate and administer the appropriate IL-1/FGF 1 inhibitor, e.g., a copper chelator (e.g., TTM), to a mammal. Indeed, the successful administration of a copper chelator has been extensively reduced to practice as exemplified herein. However, the present invention is not limited to any particular method of administration or treatment regimen. This is especially true where it would be appreciated by one skilled in the art, equipped with the disclosure provided herein, including the extensive reduction to practice using an art-recognized model of vessel injury, that methods of administering a copper chelator can be readily determined by one of skill in the pharmacological arts.

More specifically, the data disclosed herein demonstrate non-traditional export of IL-1 FGF1 mediates or is correlated with cell proliferation, macrophage infiltration, extracellular matrix deposition, restenosis, neointima formation, increase I/M ratio, and the like, and that such export can be inhibited using a copper chelator. Accordingly, based upon the disclosure provided herein, the skilled artisan would appreciate that a copper chelator can be used to treat these various diseases.

As used herein, the term “pharmaceutically-acceptable carrier” means a chemical composition with which an appropriate IL-1 release-inhibitor may be combined and which, following the combination, can be used to administer the appropriate IL-1 release inhibitor, e.g., a copperchelator, to a mammal.

The pharmaceutical compositions useful for practicing the invention may be administered to deliver a dose of between about 0.1 ng/kg/day and 100 mg/kg/day.

Pharmaceutical compositions that are useful in the methods of the invention may be administered systemically in oral solid formulations, ophthalmic, suppository, aerosol, topical or other similar formulations. In addition to the appropriate IL-1 release inhibitor, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate IL-1 release inhibitor according to the methods of the invention.

Compounds which are identified using any method described herein as potential useful compounds for treatment and/or prevention of a disease of interest can be formulated and administered to a mammal for treatment of the diseases disclosed herein are now described.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats and dogs, and birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

The present invention also includes a method of inhibiting macrophage infiltration following vessel injury in a mammal. The method comprises administering an effective amount of a copper chelator to the mammal. This is because, as more fully set forth previously elsewhere herein, decreasing the level of bioavailable copper in a cell inhibits the non-traditional export of pro-inflammatory cytokines, e.g., IL-1 and FGF1, such that various cell processes are inhibited, including, but not limited to, macrophage infiltration at the site of inflammation and/or vessel injury. Therefore, where a disease or condition is mediated by macrophage infiltration, administration of a copper chelator which reduces the amount of bioavailable copper in a cell, treats the disease or condition since the macrophage infiltration is inhibited.

One skilled in the art would also understand, once armed with the teachings provided herein, that the macrophage infiltration can be associated with inflammation, and the present invention includes methods of treating a disease or disorder mediated by macrophage infiltration where such infiltration is associated with inflammation.

The skilled artisan would appreciate, based upon the disclosure provided herein, that an “effective amount” of a copper chelator, as the term is used herein, means an amount that detectably reduces the level of copper in a cell. Such amount includes, but is not limited to, an amount of copper chelator sufficient to mediate any detectable decrease in the level of serum ceruloplasmin, since the level of ceruloplasmin is correlated to the level of bioavailable copper in cell. The skilled artisan would appreciate, based upon the disclosure provided herein, that the level of copper in a cell, or the level of bioavailable copper, can be assessed using a wide variety of methods well-known in the art and that the present invention is not limited to assessment of ceruloplasmin levels as the only measure of copper in a cell. Rather, assessing the level of ceruloplasmin is only one method of assessing the level of bioavailable copper and the invention is in no way limited to this, or any other, particular method. Further, the skilled artisan, based upon the disclosure provided herein, would understand that an effective amount of a copper chelator mediates, in turn and inter alia, a detectable decrease in level of release of a cytokine (e.g., FGF1 and IL-1) from the cell.

The invention includes a method of inhibiting cell proliferation associated with arterial wall injury. The method comprises administering an effective amount of a copper chelator to a mammal. This is because, as demonstrated and discussed previously elsewhere herein, administration of a copper chelator to a mammal, mediating a decrease in the level of bioavailable copper in a cell in the mammal, inhibits the non-traditional export (i.e., protein export that is not mediated by the ER-Golgi protein export mechanism) of a leader-less pro-inflammatory cytokine (e.g., IL-1, FGF1, and the like), such that cell proliferation at the site of vessel injury is decreased relative to the cell proliferation detected at the injury in the absence of the copper chelator. Thus, one skilled in the art, based upon the disclosure provided herein, would appreciate that cell proliferation resulting from vessel injury in a mammal can be inhibited by administering a copper chelator to the animal, where the chelator reduces the level of bioavailable copper thereby inhibiting non-traditional export of IL-1 and FGF1.

More specifically, the method provides the inhibition of cellular proliferation where the cells are vascular smooth muscle cells. Preferably, the copper chelator is TTM and the vessel injury is caused by balloon angioplasty. These methods are useful for the treatment and prevention of restenosis following balloon angioplasty. These methods are particularly useful in light of the high degree of mortalilty and morbidity associated with cell proliferation and associated restenosis following such procedures. However, the present invention is not limited to treatment of any particular disease or condition mediated by cell proliferation associated with any particular type of vessel injury. Rather, the proliferation of vascular SMCs as a result of balloon angioplasty are but one example of the successful use of the present invention to prevent restenosis in an art-recognized non-human animal model for studing such conditions.

The invention includes a method of inhibiting secretion of extracellular matrix following arterial wall injury in a mammal. The method comprises inhibiting non-traditional export of at least one of FGF-1 and IL-1 from a cell at the site of the injury, where the export is inhibited by administering an effective amount of a copper chelator to the mammal. This is because, as demonstrated by the data disclosed herein, inhibiting IL-1 and/or FGF1 release following arterial wall injury by administering a copper chelator to a mammal, inhibited secretion of extracellular matrix. This method is useful in that secretion of extracellular matrix following vessel injury is associated with and/or mediates deleterious effects and is associated with restenosis at the site of injury. Therefore, the present invention provides an important novel method for preventing deposition of extracellular matrix following vessel injury and the deleterious effects associated with such deposition.

The present invention also provides a method of inhibiting neointimal thickening associated with arterial wall injury in a mammal. The method comprises inhibiting non-traditional export of at least one of FGF-1 and IL-1 from a cell at the site of injury by administering an effective amount of a copper chelator to the mammal. This is because, as more fully set forth previously elsewhere herein, the data disclosed herein demonstrate that copper chelation inhibits the non-traditional export of pro-inflammatory cytokines, e.g., thereby inhibiting, among other things, neointimal thickening in an art-recognized animal model of vessel injury. Thus, the invention encompasses a method of inhibiting neointimal thickening by administering to an animal in need thereof, an effective amount of a copper chelator (e.g., an amount sufficient to detectably decrease the level of bioavailable copper in a cell such that there is a detectable decrease in the level of export of IL-1 and/or FGF1 release from the cell in response to stress), thereby inhibiting neointimal thickening associated with arterial wall injury.

The invention also includes a method of inhibiting adventitial angiogenesis associated with arterial wall injury in a mammal. The method comprises inhibiting non-traditional export of at least one of FGF-1 and IL-1 from a cell at the site of the injury by administering an effective amount of a copper chelator to the mammal. This is because, as more fully set forth previously herein, decreasing the level of bioavailable copper in a cell, such as by, e.g., using a copper chelator such as TIM, inhibits export of at least one of FGF-1 and IL-1 from the cell thereby inhibiting adventitial angiogenesis in response to vessel injury. More specifically, the data disclosed herein, which demonstrate extensive reduction to practice of the invention, support that administration of a copper chelator to a mammal in an art-recognized animal model, inhibits adventitial angiogenesis associated with arterial wall injury.

As more fully set forth elsewhere herein, the skilled artisan, armed with the teachings of the present invention, would understand that the invention encompasses use of a wide plethora of copper chelating compounds to reduce the level of bioavailable copper in a cell. Further, one skilled in the art would also appreciate, based upon the disclosure provided herein, that the dose, route of administration, and treatment regimen can be readily determined using methods well known in the pharmaceutical arts, such as those exemplified elsewhere herein in an art-recognized animal model. Such methods are known to the skilled artisan and are encompassed herein.

C. Methods of Identifying Useful Compounds

The invention includes a method of identifying a compound useful for inhibiting adventitial angiogenesis associated with arterial wall injury in a mammal. The method comprises contacting a cell with a compound and comparing the level of release of a leader-less pro-inflammatory cytokine by the cell in response to temperature stress with the level of release of the same cytokine from an otherwise identical cell that not contacted with the compound in response to the temperature stress. A decrease in the level of release of the cytokine by the cell contacted with the compound when compared with the release by the control, otherwise identical cell not so contacted, indicates that the compound is useful for inhibiting adventitial angiogenesis associated with arterial wall injury in a mammal.

This is because, as more fully set forth previously elsewhere herein, inhibition of release of a leader-less pro-inflammatory cytokine, which would otherwise be released in response to stress, prevents, inter alia, adventitial angiogenesis, extracellular matrix deposition, cell proliferation, macrophage infiltration, restenosis, neointimal thickening, and the like. This, in turn, is because non-traditional release of such leader-less proteins from a cell in response to stress is mediated by, among other things, copper-dependent formation of a complex which mediates the release of the cytokines. Thus, as discussed more fully elsewhere herein, reducing the level of bioavailable copper in a cell by, inter alia, copper chelation, inhibits the formation of the complex and, therefore, the release of the cytokine (e.g., FGF-1 and IL-1) from the cell. Inhibition of release of the cytokine then inhibits a variety of cytokine-mediated events, such as, but not limited to, adventitial angiogenesis.

The invention further includes a compound identified by this method, since such a compound would be a useful potential therapeutic for treatment and prevention of, among other things, adventitial angiogenesis, restenosis, macrophage infiltration, neointima formation, cell proliferation, deposition of extracellular matrix, and the like, following injury to a blood vessel.

Similarly, the invention includes methods of identifying a compound useful for treating or inhibiting restenosis, macrophage infiltration, neointima formation, cell proliferation, deposition of extracellular matrix, and the like, following injury to a blood vessel. The methods comprise contacting a cell with a compound and comparing the level of release of a leader-less pro-inflammatory cytokine (e.g., FGF-1 and IL-1) from the cell with the level of release of the cytokine from an otherwise identical cell not contacted with the compound. The skilled artisan would appreciate, based upon the disclosure provided herein, that a decrease in the level of release of the cytokine (e.g., FGF-1 and IL-1) from a cell contacted with the compound compared with the level of release from the identical cell not contacted with the compound indicates that the compound is useful for treatment or prevention of, among other things, restenosis, macrophage infiltration, neointima formation, cell proliferation, deposition of extracellular matrix, and the like, following injury to a blood vessel. This is because, as more fully set forth elsewhere herein, inhibiting the release of the cytokine inhibits these processes such that inhibitors of the release are useful potential therapeutics for treatment or prevention of these conditions.

II. Kits

The invention encompasses various kits relating to inhibiting release of a leader-less pro-inflammatory cytokine, which are useful, because, as disclosed elsewhere herein, inhibiting such release provides methods of treating or preventing adventitial angiogenesis, extracellular matrix deposition, cell proliferation, macrophage infiltration, restenosis, neointimal thickening, and the like, in a mammal. Thus, in one aspect, the invention includes a kit for inhibiting the release of IL-1 from a cell. The kit comprises an effective amount of an inhibitor of IL-1 release from a cell. The kit further comprises an applicator and an instructional material for the use thereof to be used in accordance with the teachings provided herein.

The invention also includes various kits which comprise a IL-1 release inhibitor comprising, e.g., a copper chelator and a compound, such as a fragment of S100A13, preferably, a S100A13 BR, as well as a nucleic acid encoding such a peptide, an applicator, and instructional materials which describe use of the compound to perform the methods of the invention. This is because, as demonstrated by the data disclosed herein, inhibition of the interaction of IL-1 with, for instance, a copper chelator and/or S100A13 inhibits release of IL-1 from a cell thereby inhibiting a variety of processes, including, but not limited to, restenosis, macrophage infiltration, neointima formation, cell proliferation, deposition of extracellular matrix, and the like, following injury to a blood vessel. Therefore, one skilled in the art, armed with the teachings provided herein, would appreciate that release of IL-1 from a cell can be inhibited by administering to the cell an inhibitor of such release, including, but not limited to, a copper chelator and a fragment of S100A13. The skilled artisan would further appreciate, in light of the disclosure provided herein, that various inhibitors of IL-1 release can be administered to a cell, either in concert or serially, to inhibit release of IL-1 from the cell (e.g., several copper chelators can be administered simultaneously, along with, for instance, a fragment of S100A13). Thus, the skilled artisan would understand, based upon the disclosure provided herein, that the invention encompasses use of various IL-1 release inhibitors, either together or administered temporally in a serial manner, to inhibit release of IL-1 from a cell.

The invention includes a kit for inhibiting cell proliferation associated with arterial wall injury. The kit comprises an effective amount of a copper chelator, an applicator, and instructions for the use of the kit. This is useful because, as demonstrated and discussed previously elsewhere herein, administration of a copper chelator to a mammal mediates a decrease in the level of bioavailable copper in a cell in the mammal, which in turn inhibits the non-traditional export of a leader-less pro-inflammatory cytokine (e.g., IL-1, FGF1, and the like), such that cell proliferation at the site of vessel injury is decreased relative to the cell proliferation detected at the injury in the absence of the copper chelator. Thus, one skilled in the art, based upon the disclosure provided herein, would appreciate that cell proliferation resulting from vessel injury in a mammal can be inhibited by administering a copper chelator to the animal, where the chelator reduces the level of bioavailable copper thereby inhibiting non-traditional export of IL-1 and FGF1. Moreover, the kit comprises an applicator and an instructional material for the use of the kit. These instructions simply embody the examples provided herein.

The kit can further comprise a pharmaceutically-acceptable carrier and the copper chelator is provided in an appropriate amount as set forth elsewhere herein. Further, the route of administration and the frequency of administration are as previously set forth elsewhere herein.

Although exemplary kits are described below, the contents of other useful kits will be apparent to the skilled artisan in light of the present disclosure. Each of these kits is included within the invention.

### EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

### Example 1

### Stress-Induced Release of Pro-Inflammatory Cytokine Interleukin 1 Alpha is Ci²⁺-Dependent

Copper is involved in the promotion of angiogenic and inflammatory events in vivo and although recent clinical data has demonstrated a potential therapeutic role for Cu²⁺-chelators in the treatment of cancer in humans, the mechanism for this activity remains unknown. Since FGF1 and IL1α exhibit similar crystallographic structures (Zhu et al., 1991, Science 251:90-93; Graves et al., 1990, Biochemistry 29:2679-2684), both FGF1 and IL1α are released in response to stress (Tarantini, et al. 2001, J. Biol. Chem. 276:5147-5151), and the Cu²⁺ chelator, TTM, has been shown to be effective in the clinical management of solid tumor growth (Brewer et al., 2000, Clin. Cancer Res. 6:1-10; Cox et al., 2001, Laryngoscope 111:696-701; Merajver et al., 2001, personal communication and submitted to Nature Med.), it was examined whether the release of IL1α could be modified by the expression of S100A13 and whether intracellular Cu²⁺ was involved in the release of IL1α in response to heat shock.

The data disclosed herein demonstrate that like the signal peptide-less prototype members of the FGF gene family, the leader-less IL1 prototypes are also Cu²⁺-binding proteins. In addition, the data disclosed herein demonstrate that the appearance of extracellular IL1α in response to cellular stress involves the intracellular function of the Cu²⁺-binding protein, S100A13, and is repressed by the Cu²⁺ chelator, tetrathiomolybdate (TTM). In addition, the data disclosed herein demonstrate the expression of a S100A13 mutant lacking a sequence novel to this gene product functions as a dominant-negative repressor of IL1α release whereas the expression of wild type S100A 13 functions to eliminate the requirement for stress-induced transcription.

The materials and methods used in the experiments presented in this Example are now described.

Material and Methods

Cell Lines and Recombinant Proteins

Stable NIH 3T3 cell transfectants were generated accordingly with the following cDNA constructs cloned into pMEXneo (Tarantini, et al. 2001; Tarantini, et al. 1995; Tarantini, et al. 1998; LaVallee, et al. 1998; Carreira, et al. 1998; Landriscina, et al., 2001): human S100A13 with an amino-terminal fusion to a 6 Myc Tag (Myc-S100A13) (Landriscina, et al., 2001); human Myc-S100A13ΔBR (Landriscina, et al., 2001); human mIL1α (residues 113-271); human mIL1α with a carboxy-terminal fusion to a βGalactosidase tag (mIL1α-βGal) (Tarantini, et al. 2001); and pIL1α (residues 1-271) with a carboxy-terminal fusion to βGalactosidase (pIL1α-βGal) (Tarantini, et al. 2001). Recombinant human mIL1α was provided by Hoffmann-LaRoche. Recombinant human S100A13, as well as S100A13ΔBR, a S100A13 construct with the basic residue rich domain (residues 88 to 98) deleted, were generated as previously described (Landriscina, et al., 2001).

Ultracentrifugational Analysis

IL1α and either S100A13 or S100A13ΔBR were incubated at molar ratios (IL1α:S100A13) of 1:1, 1:5, and 1:10 in phosphate-buffered saline (PBS) either in the presence or absence of 1 mM CuCl₂ for 30 minutes at 42 C followed by centrifugation at 280,000×g for 18 hours at 4 C and resolution of the pellet fractions by S100A13 immunoblot analysis was performed as described by Landriscina et al. (2001).

Ammonium Sulphate Fractionation and Analysis of Protein Interactions in Heat Shock-Conditioned Media

IL1α and S100A13 were incubated as described for ultracentrifugational analysis, followed by incubation of the reactions in 100% (NH₄)₂SO₄ at 4 C for 30 minutes, centrifugation at 10,000×g for 30 minutes, and resolution of the pellet and supernatant fractions by S100A13 immunoblot analysis as described in Landriscina et al. (2001).

NIH 3T3 cell transfectants were grown to 70-80% confluency and prior to temperature stress, the cells were washed with serum-free DMEM. The heat shock was performed as previously described (Tarantini et al., 1995) in serum-free DMEM for 110 min at 42° C. Control cultures were incubated at 37° C. in serum-free DMEM. Two independent clones from each transfection were evaluated with similar results. For the analysis of the release of Myc-S100A13, mature IL1α-βgal and precursor IL1α-βGal, DTT-treated conditioned media (2 hours at 37° C.) and cell lysates from the appropriate NIH 3T3 cell transfectants were prepared and divided into two portions, one of which was processed as described for S100A13 immunoblot analysis of the Myc reporter sequence (Landriscina, et al. 2001) and the other for mature IL1α-βGal and precursor IL1α-βGal immunoblot analysis (Tarantini, et al. 2001). Briefly, one portion was concentrated and immunoprecipitated with an anti-IL1α antibody for the evaluation of mature IL1α-βGal release and the second portion was adsorbed to heparin-Sepharose and eluted at 1.5 M NaCl for evaluation of Myc-S100A13 release. Immunoprecipitated and eluted proteins were resolved by 8% and 12% acrylamide SDS-PAGE, respectively, and evaluated by either IL1α (Tarantini, et al. 2001) or Myc (Landriscina, et al., 2001) immunoblot analysis. The activity of lactate dehydrogenase in conditioned media was utilized as an assessment of cell lysis in all experiments, as previously reported (Tarantini et al., 2001). For the analysis of the heparin affinity of mature IL1α-βGal released from Myc-S100A13 and mature IL1α-βGal NIH 3T3 cell cotransfectants, ammonium sulfate saturation was performed as described (Landriscina et al., 2001). The effects of actinomycin D (Sigma Chemical Co., St. Louis, Mo.), cyclohexamide (Sigma) and tetrathiomolybdate (Sigma-Aldrich) on IL1α release were evaluated as previously reported (LaVallee et al., 1998).

The results of the experiments presented in this Example are now described.

IL1α is a Cu²⁺-Binding Protein

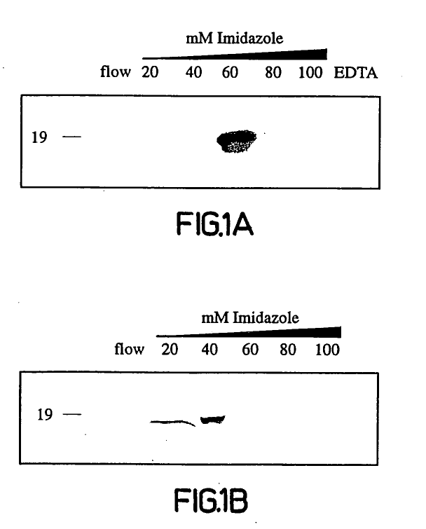

Unlike FGF1 (Jaye et al., 1986; Abraham et al., 1986), human IL1α contains a single Cys residue (Dinarello, 1994; Krakauer, 1986; Dinarello, 1998), which is not conserved among species, yet crystallographic evidence suggests the presence of three histidine residues which are accessible to solvent (Graves et al., 1990). Since histidine residues are involved in Cu²⁺-binding (Kwiatkowski et al., 1977; Kingston et al., 1979), the ability of IL1α to bind immobilized Cu²⁺ was evaluated. As shown in FIG. 1A, recombinant human IL1α is an avid Cu²⁺-binding protein, requiring 60 mM imidazole for elution. Similar elution data were also obtained using recombinant human IL1β. The Cu²⁺-binding character of the IL1 prototypes was quite surprising since FGF1, S100A13 and p40 Syt1 elute from immobilized Cu²⁺ at 40 mM imidazole (Landriscina, et al. 2001).

In order to demonstrate whether the form of IL1α released in response to temperature stress exhibited similar Cu²⁺-binding attributes, IL1α NIH 3T3 cell transfectants were assessed for their ability to release IL1α as a Cu²⁺-binding protein in response to stress. IL1α immunoblot analysis of cell culture media conditioned by heat shock (FIG. 1B), but not cell culture media conditioned at 37 C, exhibited the presence of IL1α as a Cu²⁺-binding protein. Surprisingly, the imidazole elution character of IL1α was altered and, unlike the recombinant polypeptide, it was eluted at 40 mM imidazole (FIG. 1B).

IL1α Utilizes S100A13 for Stress-Induced Release

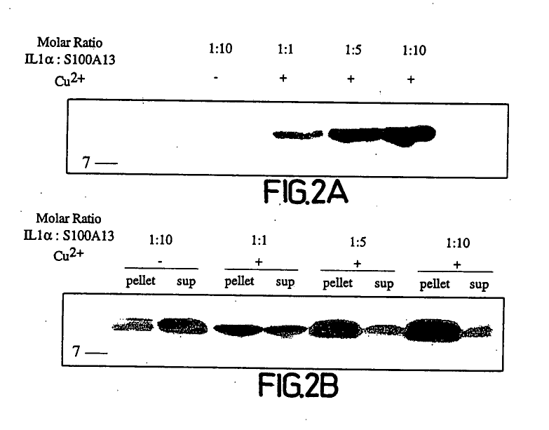

Because FGF1 utilizes the function of the S100A13 gene product to facilitate its release in response to stress (Landriscina, et al., 2001), it was examined whether IL1α could also utilize S100A13. In order to address this premise, the ability of the recombinant human forms of IL1α and S100A13 to interact and form a Cu²⁺- and molar ratio-dependent multiprotein aggregate which would be susceptible to ultracentrifugation was examined. As shown in FIG. 2A, S100A13 was present in the pellet fraction following centrifugation at 280,000×g for 18 hours only when incubated with IL1α and only in the presence of Cu²⁺. In addition, the level of S100A13 present in the pellet fraction increased as a function of the IL1α to S100A13 molar ratio with a maximum between a molar ratio of 1:5 to 1:10, suggesting that IL1α and S100A13 can interact in a Cu²⁺-dependent manner.

Because the S100 gene family was named for their solubility in 100% (NH₄)₂SO₄ (Moore, 1965, Biochem. Biophys. Commun. 19:739-744) and IL1α is susceptible to salt fractionation (Hirano et al., 1981, J. Immunol. 126:517-522; Mizel et al., 1981, J. Immunol. 126:834-837), IL1α and S100A13 were incubated with saturated (NH₄)₂SO₄ at varied molar ratios in the presence and absence of Cu²⁺. Following centrifugation, the supernatant fraction was analyzed using S100A13 immunoblot analysis. As depicted in FIG. 2B, S100A13 was present in the pellet fraction in a Cu²⁺- and IL1α-dependent manner and its presence in the pellet fraction was a function of the IL1α to S100A13 molar ratio with a maximum occurring between a molar ratio of 1:5 and 1:10.

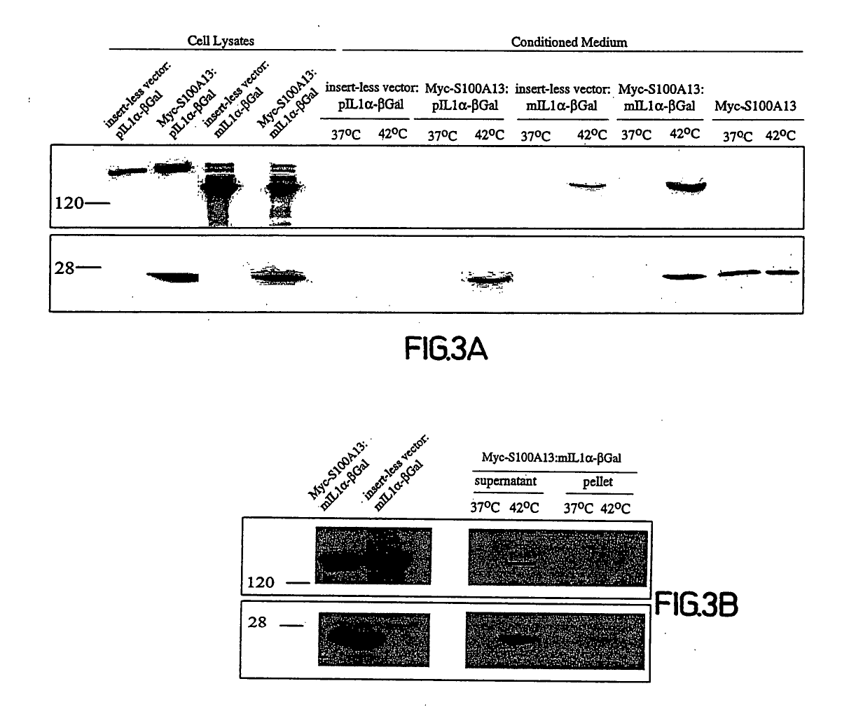

Since IL1α and S100A13 are able to interact in a cell-free system in a Cu²⁺-dependent manner, it was examined whether the expression of the precursor and mature forms of IL1α can also repress the constitutive release of intracellular S100A13. Thus, S100A13 containing an NH₂-terminal Myc epitope tag was stably transfected into precursor IL1α-βGal and mature IL1α-βGal NIH 3T3 transfectants (Tarantini, et al. 2001) and the cotransfectants were either maintained at 37 C for 2 hours or subjected to heat shock. Insert-less vector and mature IL1α-βGal, as well as insert-less vector and precursor IL1α-βGal, NIH 3T3 cell cotransfectants served as a control. As shown in FIG. 3A, the expression of either the precursor or the mature form of IL1α was able to repress the constitutive release of Myc-S100A13 at 37 C. In addition, the data demonstrate the presence of Myc-S100A13 in medium conditioned by heat shock from Myc-S100A13 and precursor IL1α NIH 3T3 cell cotransfectants (FIG. 3A), suggesting that S100A13 can gain access to the extracellular compartment independent of IL1α release.

Unlike FGF1 (Maciag et al. 1984, Science 225:932-935), the mature form of IL1α does not bind immobilized heparin (Tarantini et al. 2001, J. Biol. Chem. 276:5147-5151), yet S100A13 has been characterized as a heparin-binding protein (Landriscina et al. 2001, J. Biol. Chem. 276:22544-22552). Thus, if IL1α and S100A13 were present in the extracellular compartment as a complex, IL1α should gain both heparin affinity and solubility following 100% (NH₄)₂SO₄ fractionation as a result of its association with S100A13. In order to evaluate this premise, the Myc-S100A13 and IL1α-βGal NIH 3T3 cell cotransfectants were subjected to heat shock, and the conditioned medium was subjected to 100% (NH₄)₂SO₄ fractionation. Pellet and supernatant fractions were adsorbed to immobilized heparin, eluted with 1.5 M NaCl and the presence of IL1α and S100A 13 analyzed by IL1α and Myc immunoblot analysis. As shown in FIG. 3B, the Myc-S100A13 and IL1α-βGal NIH 3T3 cell cotransfectants were able to release Myc-S100A13 and IL1α-βGal as a heparin-binding complex and while both proteins were present in the supernatant fraction following 100% (NH₄)₂SO₄ fractionation, S100A13 was also present in the pellet fraction. Because the Cu²⁺-dependent cell-free system (FIG. 2B) also demonstrated the presence of IL1α and S100A13 in the pellet and supernatant fractions at an equimolar concentration, without wishing to be bound by any particular theory, these data suggest that IL1α-βGal and Myc-S100A13 can be present in the extracellular compartment at a 1:1 molar ratio.

A S100A13 Mutant Lacking the Basic Residue-Rich Domain is a Dominant Negative Regulator of Stress Induced IL1α Release

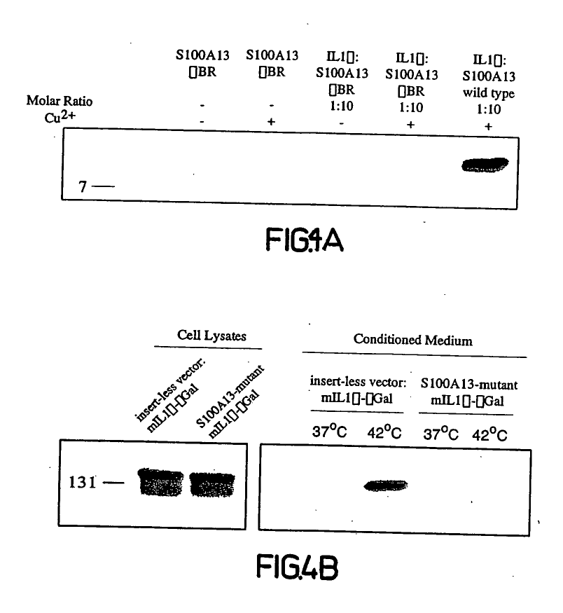

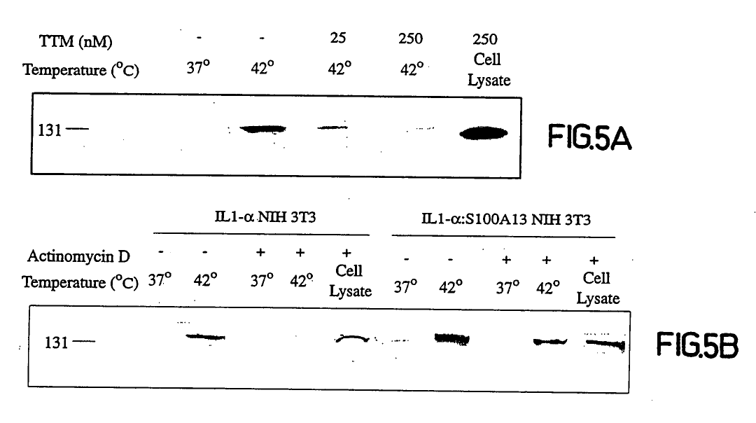

Because the data suggested that IL1α and S100A13 can associate, the domain in S100A13 responsible for this association was assessed. The basic residue (BR)-rich domain at the carboxy-terminus of S100A13 (Wicki, et al. 1996) was examined. Thus, the last eleven residues in S100A13 were deleted and the ability of the recombinant form of S100A13ΔBR to associate in a Cu²⁺-dependent manner with IL1α in a cell-free system was assessed. As shown in FIG. 4A, the S100A13ΔBR failed to precipitate in the presence of Cu²⁺ and IL1α. Furthermore, like S100A13 (Landriscina, et al. 2001), the recombinant form of S100A13ΔBR eluted from immobilized Cu²⁺ at 40 mM imidazole. In addition, a deletion mutant of S100A13ΔBR containing a multiple Myc epitope tag (Myc-S100A13ΔBR) was produced and used to produce IL1α-βGal NIH 3T3 cell co-transfectants. The ability of the IL1α-βGal and Myc-S100A13ΔBR NIH 3T3 cell cotransfectants to release IL1α-βGal in response to temperature stress was then evaluated. As shown on FIG. 4B, IL1α-βGal was not detected in media conditioned by heat shock from IL1α-βGal and Myc-S100A13ΔBR NIH 3T3 cell cotransfectants.

The Cu²⁺ Chelator, Tetrathiomolybdate (TTM), Inhibits the Stress-Induced Release of IL1α

The ability of Cu²⁺ to mediate the interaction between IL1α and S100A 13 suggests that intracellular Cu²⁺ is involved in the regulation of the stress-induced release of IL1α. In order to examine this premise, the ability of the Cu²⁺ chelator, TTM to repress the release of IL1α in response to heat shock was assessed. As shown in FIG. 5A, TTM inhibited release of IL1α at 250 nM and this concentration is consistent with the concentration of TTM used in clinical trials (Brewer et al. 2000, Clin. Cancer Res. 6:1-10; Cox, et al. 2001, Laryngoscope 111:696-701; Merajver et al., personal communication and submitted to Nature Med., 2001). Similar results were also observed for the inhibition of FGF1 release in response to stress.

It was also examined whether the expression of S100A13 as a Cu²⁺-binding protein could overcome the requirement for heat shock-induced transcription by examining the ability of actinomycin D to repress the export of IL1α into the extracellular compartment when expressed in a S100A13 background. As shown in FIG. 5B, while actinomycin D was able to repress the release of IL1α-βGal from insert-less vector and IL1α-βGal NIH 3T3 cell cotransfectants, actinomycin D was unable to repress the export of IL1α-βGal from Myc-S100A13 and IL1α-βGal NIH 3T3 cell cotransfectants in response to heat shock (FIG. 5B). However, the introduction of TTM into this system was able to repress the release of IL1α-βGal in response to temperature stress and similar results were also obtained when cyclohexamide was used to inhibit translation.

Divalent copper is becoming increasingly recognized for its role in many normal physiological and pathological processes. Indeed, while both S100A13 (Landriscina, et al. 2001, J. Biol. Chem. 276:22544-22552) and FGF1 (Engleka and Maciag, 1992, J. Biol. Chem. 267:11307-11315) have been characterized as Cu²⁺-binding proteins, the prototype members of the IL1 gene family have not been characterized, until now, as Cu²⁺-binding proteins, despite a high degree of structural conservation between the FGF and IL1 prototypes (Zhang, et al. 1991, Graves, et al. 1990) as well as the presence of three solvent accessible histidine residues (Graves, et al. 1990), which are conventionally regarded as being important for the binding of proteins to copper (Kwiatkowski, et al. 1977; Kingston, et al. 1979). Surprisingly, while recombinant IL1α eluted from the Cu²⁺-affinity column with 60 mM imidazole, IL1α obtained from medium conditioned by heat shock eluted with 40 mM imidazole. This change in elution character between the recombinant and released forms of IL1α is interesting, since the elution character of the released form of IL1α present in medium conditioned by temperature stress resembles the elution character of the Cu²⁺-induced FGF1, p40 Syt1 and S100A13 multiprotein aggregate (Landriscina, et al. 2001) suggesting, without wishing to be bound by any particular theory, that extracellular IL1α can be associated with one of the proteins involved in the regulation of the stress-induced release of FGF1. Indeed, this suggestion is consistent with the data disclosed herein demonstrating that mIL1α and S100A13 are able to form a multiprotein Cu²⁺-dependent complex which alters the sedimentation and solubility of S100A13 at 100% (NH₄)₂SO₄ saturation. In addition, the data disclosed herein further demonstrate that both, the mature and the precursor forms of IL1α, can access intracellular S100A13.

The data disclosed herein also suggest that the carboxy-terminal basic-rich (BR) domain of S100A13 can mediate the interaction with the mature form of IL1α. The carboxy-terminus of other S100 gene family members has been implicated in mediating their ability to interact with proteins (Schafer and Heizmann, 1996, Trends in Biochem. Sci. 21:134-140; Kilby et al., 1996, Structure 4:1041-1052; Pozdnyakov et al., 1998, Biochemistry 37:10701-10708; Rety et al., 1999, Nat. Struct. Biol. 6:89-95). Interestingly, unlike other S100 gene family, S100A13 contains a nine amino acid basic residue-rich domain which is absent in other S100 gene family members (Schafer and Heizmann, 1996; Wicki et al., 1996, Biochem. Biophys. Res. Commun. 227:594-599). Thus, the data disclosed herein demonstrate, for the first time, that the stress induced interaction between the mature form of IL1α and the BR domain of S100A13 promotes the release of both proteins as a Cu²⁺-dependent complex.

It is also noteworthy that the expression of S100A13 in an IL1α background results in an attenuation of the sensitivity of the IL1α release pathway to the transcription inhibitor actinomycin D. Without wishing to be bound by any particular theory, since the transcription of the S100A13 gene is not regulated by heat shock, it is likely that the role of cellular stress in the export of the mature form of IL1α may not be due to the induction of a classical stress-mediated transcriptional response; rather, the stress response may involve the regulation of a post-translational activity which modifies S100A13. Although the nature of this putative post-translational activity is not yet known, the data disclosed herein suggest that it is possible that the oxidative character of intracellular Cu²⁺ may be involved in the regulation of this feature.

Although the copper chelator, tetrathiomolybdate (TTM), has been assessed in the management of human cancer in recent clinical trials, the molecular mechanisms that may be operating in whole tissues during copper deficiency had remained unknown. In particular, there were no reports of the potential role of copper deficiency in inhibiting immune-mediated paracrine stimulation of angiogenesis, a phenomenon that is presumed to be key to the inhibition of tumor growth in situ. The data disclosed herein demonstrate, for the first time, the unanticipated role of copper in the release of the pro-inflammatory and pro-angiogenic cytokine, IL-1α. Without wishing to be bound by any particular theory, because TTM also represses the release of FGF1, the ability of Cu²⁺ chelators to act as effective clinical anti-cancer agents can be related to their ability to limit the export of these proinflammatory and angiogenic signal peptide-less polypeptide hormones into the extracellular compartment.

The data disclosed herein demonstrate, for the first time, that the release of IL-1α can be selectively inhibited by copper chelation and/or by administering a truncated form of S100A13. More specifically, the data disclosed herein demonstrate, for the first time, that TTM and S100A13 BR can inhibit the stress-induced release of IL-1α, which can consequently prevent the infiltration of mononuclear cells laden with proangiogenic factors, like FGF1 to tumor environments, as more fully disclosed elsewhere herein. These results not only support the use of TTM for the therapeutic management of tumor diseases in mammals, but also demonstrate that an IL-1 receptor antagonist can also exhibit similar clinical potential.

### Example 2

### Restenosis and Neointimal Formation

Neointima formation associated with vascular restenosis after coronary intervention is a complex process mediated by inflammatory cytokines and growth factor activities, which regulate vascular smooth muscle cell (SMC) migration and proliferation. Since intracellular copper metabolism plays a crucial role in the stress-induced release of FGF-1 and IL-1α, which are known to be important for SMC proliferation and inflammatory cell migration, the in vivo effect of copper, using TTM, was assessed using balloon-induced neointimal formation in an art-recognized rat carotid artery model.

The materials and methods used in the experiments presented in this Example are now described.

Animals

Seventy-nine Sprague-Dawley male rats (Charles River Laboratories) weighing 350 to 450 grams, at 12-16 weeks of age, were included in the study. All rats in this study were handled according to the animal welfare regulation of the Maine Medical Center Research Institute, and the study protocol has been approved by the Animal Care and Use Committee of that institution. The rats received humane care in accordance with the animal use principles of the American Society of Physiology. All rats were maintained under identical conditions of temperature (21±1° C.), humidity (60±5%), and light/dark cycle, and had free access to normal rat chow.

Study Design

A copper chelator, ammonium tetrathiomolybdate (Sigma Aldrich), was administered daily in a dose of 10 mg/kg. The total daily amount was freshly dissolved into 45 ml water and was dispensed to the rats in the drinking water. To assess the effect of copper chelation on neointimal formation after common carotid artery denudation, TTM was given as follows: TTM administration started 2 weeks before the injury in 6 rats; 1 week before the injury in 6 rats; there was no pre-injury treatment with TTM in 5 rats. All those rats were treated with TTM for 2 weeks after the balloon injury. Five rats, which underwent the surgical procedure and were never treated with TTM, served as controls.

To assess the effect of time course of copper chelation in relation to the injury on the neointima formation, 5 additional groups of rats were studied. All of them were treated with TTM for 2 weeks before the injury, and then TTM was withheld as follows: at the day of the injury (n=6) as well as 4 days (n-5), 6 days (n=7), 8 days (n=6), and 10 days (n=6) after the balloon denudation.

In order to address the mechanisms underlying the effect of copper chelation on neointima formation, TTM was administered for 2 weeks before the balloon injury and daily after that until the animals were euthanized at the 4th and 7th day after the procedure. Accordingly, TTM-free animals euthanized 4 and 7 days after the injury were used as controls.

Surgical Procedure and Tissue Preparation

Rats were anesthetized with an intraperitoneal injection of ketamine (50 mg/kg) and xylazine (2.2 mg/kg). Angioplasty of the carotid artery was performed with a balloon embolectomy catheter as previously described. Briefly, the balloon catheter (2F Fogarty, Edwards Laboratories) was introduced through the left external carotid artery into the aorta, and the balloon was inflated. The vessel was damaged by passing an inflated balloon through the lumen three times. The catheter was rotated when pulling back. At the time of the final experiment, the animals were euthanized after anesthesia. A midline abdominal incision exposing the distal abdominal aorta was made. After retrograde cannulation of the abdominal aorta at its bifurcation with a 18-gauge intravenous catheter, the arterial tree was cleared of blood by perfusion with 100 ml of PBS (pH 7.2), followed by in vivo fixation with either 4% formaldehyde in phosphate-buffered saline (pH 7.2) or acetone/ethanol solution in 1:1 ratio. The entire left and right carotid arteries were harvested, including the aortic arch, innominate artery and left carotid bifurcation, and further immersed in the respective fixative. The injured left common carotid arteries were cut in three sections at least 4 mm long from the proximal, middle and distal part. From each study group, a part from the untreated contra-lateral right common carotid artery (control) was taken as well. The specimens were then dehydrated through a graded ethanol series, and embedded in paraffin for sectioning. Other arteries were not perfusion-fixed but were removed and immediately frozen. Three different segments of the left carotid artery were used for histological, morphometric, and immunohistochemical studies.

Histomorphometric Study

Morphometric analysis of the arterial segment was carried out in a blind manner on cross-sections stained with hematoxylin-eosin. For each animal at least 3 sections originating from the proximal, middle and distal segment of the injured vessel were quantitatively measured. Using a computerized digital microscopic planimetry algorithm (Optimas, Version 5.22), the areas within the external elastic lamina (EEL area), the internal elastic lamina (IEL area), and the luminal area were measured. Other areas were calculated as follows: medial area=EEL area−IEL area; neointimal area=IEL area−luminal area; neointima-to-media (I/M ratio=neointimal area/medial area.

Intimal cell counting was performed according a previously described method that is standard in the art. Briefly, analyses were performed on cross sections stained with hematoxylin-eosin under ×40 microscopic magnification. Random areas (encompassing 20 to 40% of the total intimal cross-sectional area) within the intima were selected, and cell nuclei were enhanced and counted after dynamic color thresholding. The average cell nuclear count within these known areas was used to calculate the cell density (cells/mm²)

Immunohistochemistry.

To evaluate S100A13, FGF1, p40 and IL-1α expression in balloon-injured arteries, paraffin-embedded specimens from the 4th, 7th and 14th day after the injury were cut into 5-μm cross sections, and mounted on glass slides. These sections were incubated in 10% hydrogen peroxide for 90 minutes to block endogenous peroxidase activity. Nonspecific binding was prevented by preincubating the sections with 5% bovine serum albumin (BSA; Sigma) in PBS. The sections were sequentially incubated with polyclonal rabbit anti-S100A13 antibody at a concentration of 1:200; polyclonal rabbit anti-FGF1 antibody at a concentration of 1:500; monoclonal mouse anti-p40 antibody at a concentration of 1:100; polyclonal rabbit anti-IL-1a antibody at a concentration of 1:50; phosphatidylserine (PS) antibody. After they were washed with PBS, the sections were incubated with anti-rabbit and anti-mouse IgG-conjugated horseradish peroxidase (Biorad) for an additional 30 minutes at room temperature. Each incubation was followed by a wash in PBS. Staining was visualized using the chromogen 0.06% 3,3′-diaminobenzidine/5% hydrogen peroxide in 0.05 mol/L Tris-HCl (pH 7.6). Control sections were incubated with nonimmune rabbit IgG at a concentration of 1:200.

Proliferating cell nuclear antigen (PCNA) analysis was used to quantify the proliferative activity of cells at the balloon injury sites, and it was performed according to Siitonen et al. Briefly, PCNA-positive cells were counted in the vessel cross sections using a standard light microscope equipped with an ocular reticule (magnification ×10) and a ×40 objective. At least 500 nuclei were counted from each slide. The sections were photographed under low power, the images were video-digitized, and stored in the image analysis system (Qwin Lite 2.2, Leica). Staining results were expressed as percentage of PCNA-immunoreactive cell nuclei. Faint diffuse nuclear staining seen in some tissue sections was not included in the PCNA score.

Macrophage migration was evaluated by immunostaining with the macrophage-specific monoclonal antibody CD11b (MAC1) in acetone/ethanol fixed sections. To quantitate the extent of macrophage invasion, the area occupied by MAC1-positive cells as a percentage of the total area of the neointima was determined. The number of SMCs in the injured artery was counted at day 4, 7 and 14 by the modified method of Prescott et al. Briefly, the cross-sections were subjected to immunohistostaining against α-smooth muscle actin using a commercially available detection system (DAKO) and counter-stained with hematoxylin. The number of nuclei that were accompanied by α-smooth muscle actin-positive cytoplasm was counted at a magnification of ×40 in 10 independent sections from each rat by an observer in a blind manner.

Serum Chemistry Assay

Copper status in mammals treated with TTM cannot be reliably followed by measuring total serum copper level because the chelated copper will still be detected. Serum ceruloplasmin, whose synthesis is directly regulated by the bio-availability of copper to the liver, is a more accurate indicator of free copper and is used as a surrogate marker of free copper status Schosinksy et al., 1974, Clin. Chem. 20:1556-1563. Serum ceruloplasmin was assessed as baseline level before the TTM-treatment, as well as on the day of the injury and on the final day. Blood (0.5 to 1 ml) was obtained from the tail vein after anesthesia was centrifuged for 10 minutes at 2000×G and the serum was frozen at −20° C. until assay. Ceruloplasmin oxidative activity was measured as described previously (Schosinsky et al., 1974, Clin. Chem. 20:1556-1563).

Statistical Analysis

All variables are expressed as mean+SEM. Student's t-test was used to exam the differences between the experimental groups. The time courses of the ceruloplasmin levels before and after treatment were compared by ANOVA for repeated measures. A value of p<0.05 was considered significant.

The results of the experiments presented in this Example are now described.

Copper Chelation Attenuates Neointima Formation after Balloon Injury

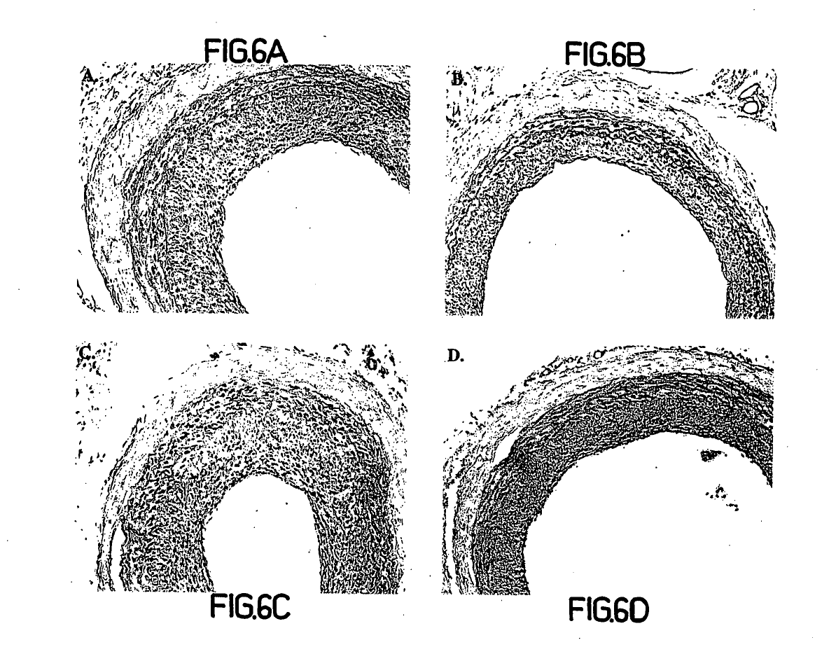

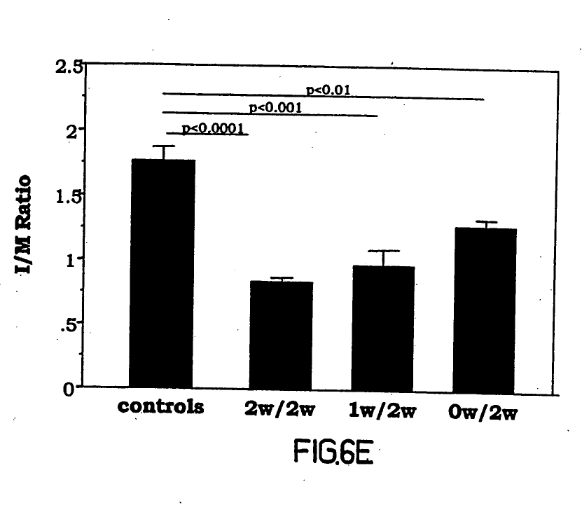

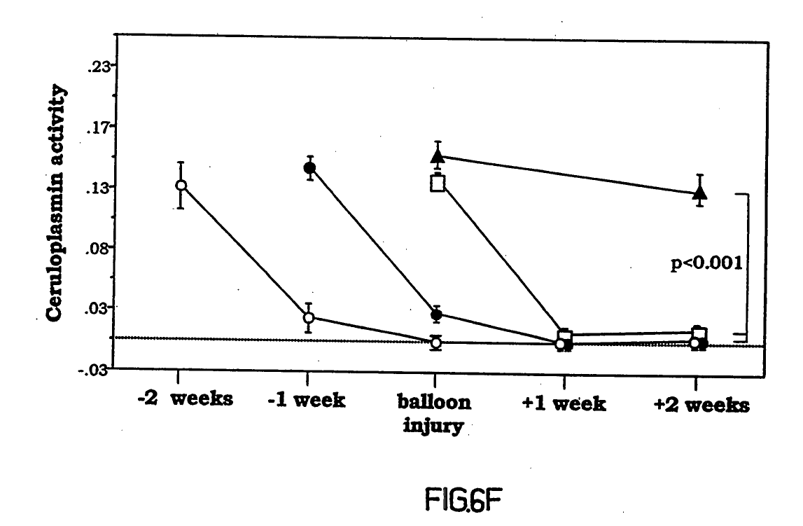

Fourteen days after balloon injury and daily treatment with TTM, the neointima formation as estimated by intima/media ratio was remarkable prevented in rats, which TTM-application started 2 weeks (n=6) or 1 week (n=6) before the injury than in the controls (n=5) as well as in those rats (n=5), which TTM application started on the same day of the balloon injury (0.83±0.006 and 0.96±0.121 versus 1.76±0.105, and 1.27±0.04 respectively, P<0.05) (FIG. 6). Thus, TTM administration before and after injury leads to up to 53% reduction in the neointima formation in the rat carotid artery.

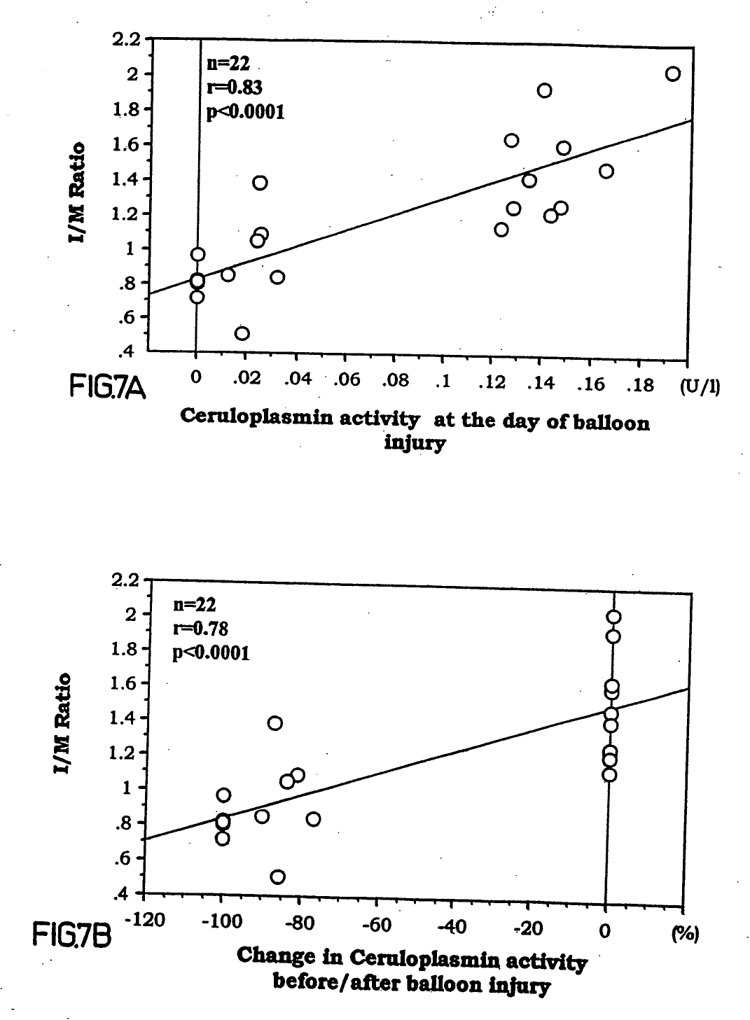

When the I/M ratio was plotted against the serum ceruloplasmin at the day of the injury or against the change in the ceruloplasmin after TTM-treatment, significant linear relations were observed (r=0.84, p<0.0001 and r=0.785, p<0.0001, respectively) (FIG. 7).

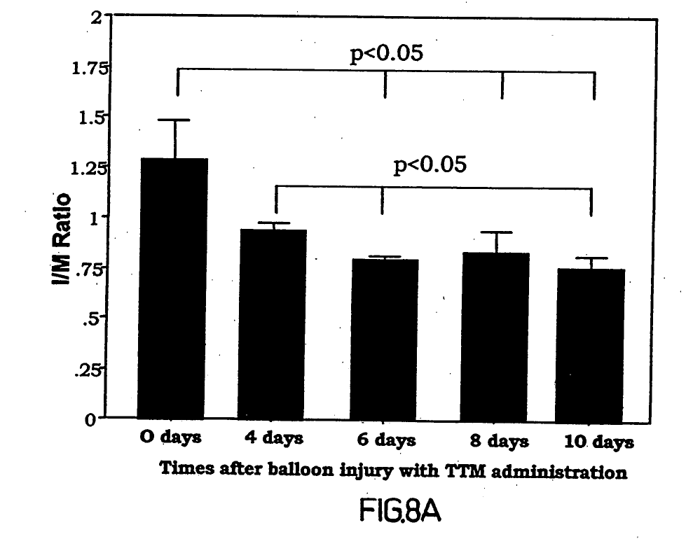

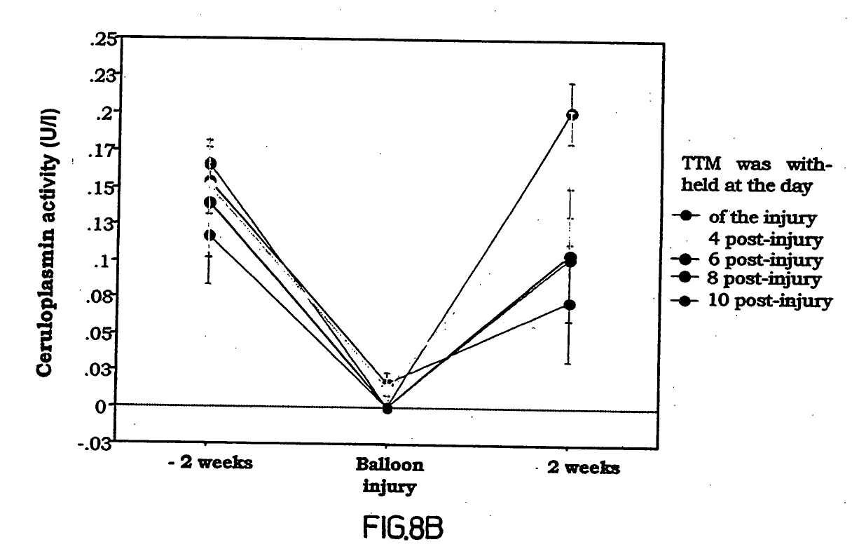

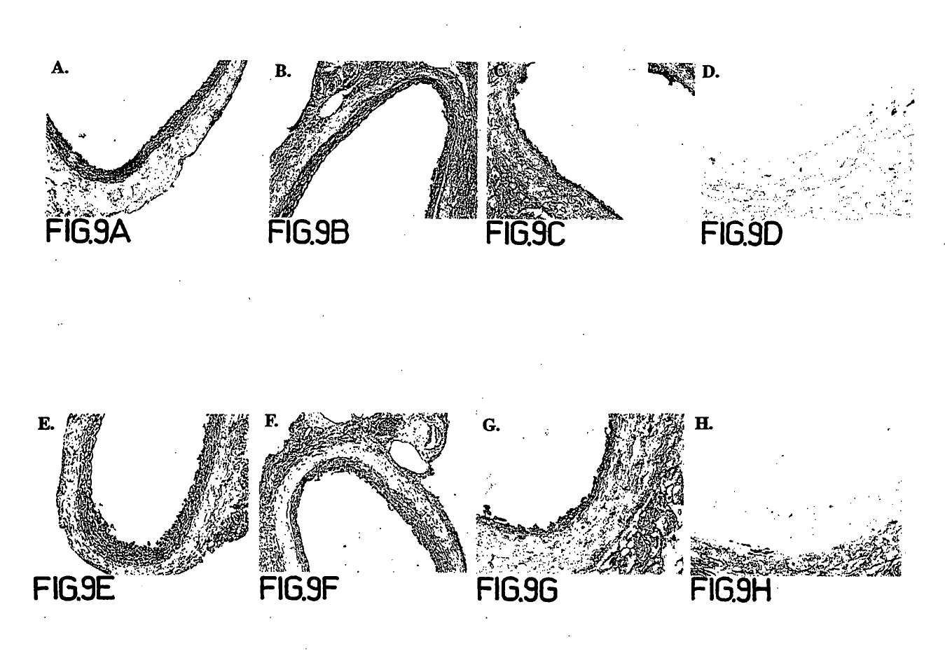

Two weeks of treatment with TTM demonstrated the best result regarding copper chelation and inhibition of neointima formation. Consequently, the effect of TTM-withholding at different times after the balloon injury was assessed. A significant decrease in intima/media ratio was observed when the TTM administration started 2 weeks before the injury and was continued for either 6, 8 or 10 days after the injury (0.76±0.06, 0.83±0.1, 0.79±0.013, respectively) as compared to the controls. However, this effect was diminished when TTM administration was stopped at the day of the injury (1.29±0.19) or 4 days after (0.93±0.0.34)(FIG. 8).

Copper Chelation Reduces Macrophages Infiltration into the Arterial Wall

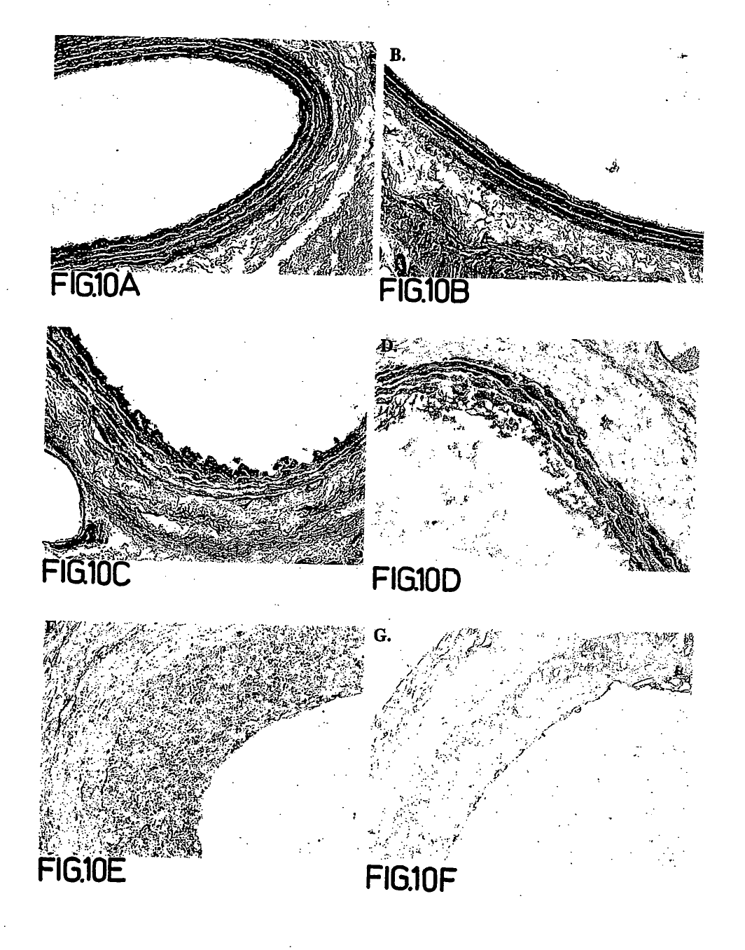

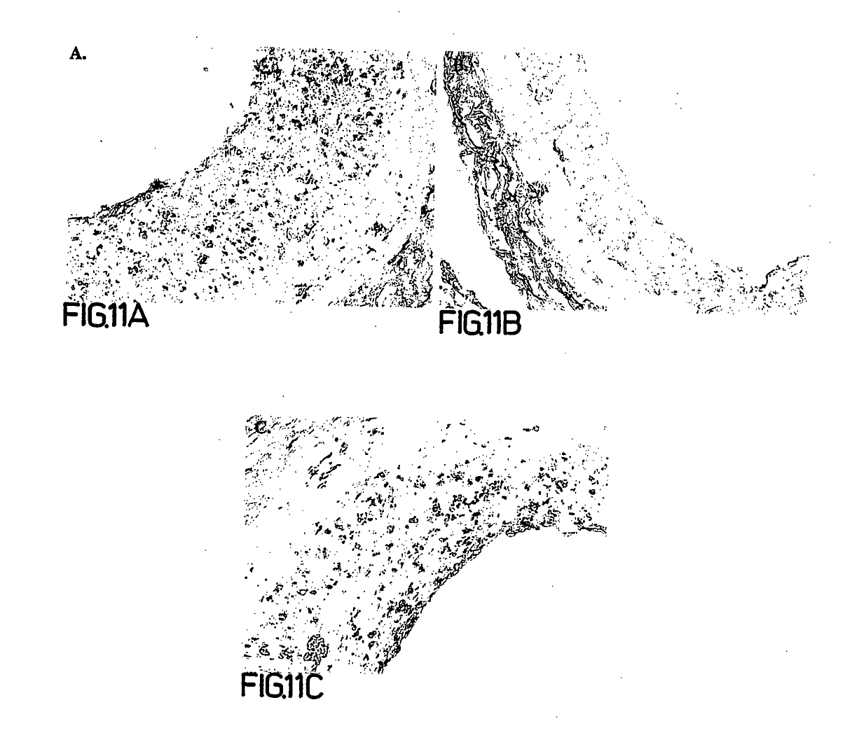

Massive macrophage infiltration was detected in the adventitia around the vessel and in the neointima 4 days after injury in the TTM-free animals (FIG. 9). By day 7 after injury, macrophages were found in the media as well, and the entire wall was filled with macrophages in those animals (FIG. 9). However, very few macrophages were present at any time point after the injury within the arterial wall in the rats treated with TTM (FIG. 9). Only a few macrophages were found in the intima at either time point. FIG. 9 depicts that 4 days after injury macrophages in the neointima were not detectably different between the control group (FIG. 9E) and the TTM-treated group (Figure A). At day 7 after injury macrophages were more pronounced in the controls (FIG. 9F) than in the TTM-treated group (FIG. 9B).

Copper Chelation Inhibits Cell Proliferation in the Injured Arterial Wall

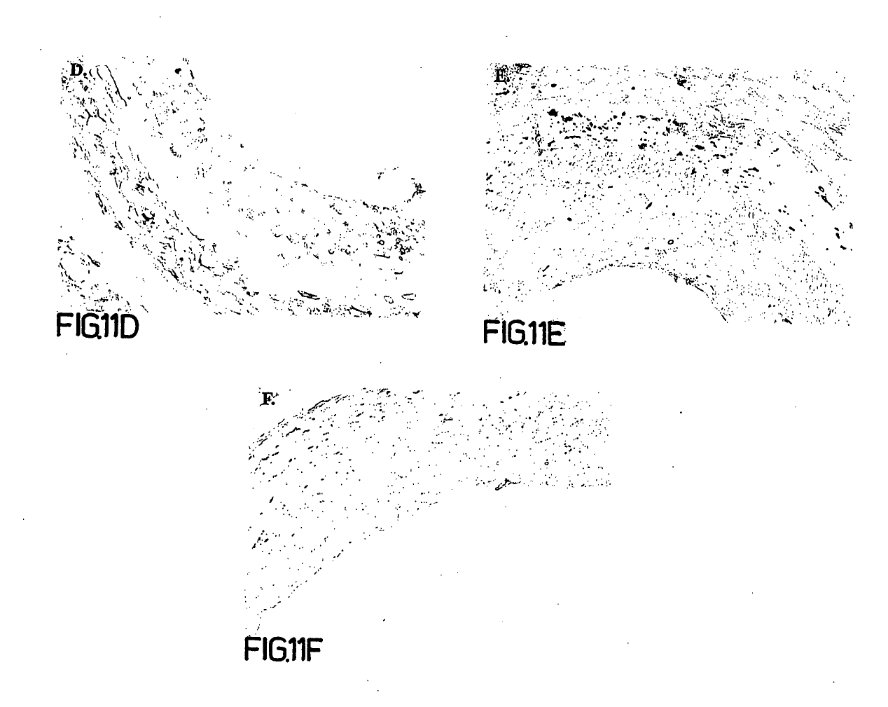

SMA-positive cells were significantly less abundant in the neointima in the TTM-treated rats when compared to the tissue obtained from TTM-free rats 7 and 14 days after the injury (FIG. 10). No significant difference was found between the groups by day 4 after the balloon injury.

PCNA is a 36-kDa acidic nuclear polypeptide that is involved in DNA synthesis as a cofactor for DNA polymerase delta. PCNA plays a critical role in the initiation of cell proliferation, and its expression is elevated almost exclusively during the S phase of the cell cycle. PCNA-positive cells were not observed in the sections of non-injured carotid arteries. Since in all injured carotid arteries analyzed, the percentage of PCNA-positive cells in the media was <1%, only the percentages of positively stained cells in the neointima were used to compare the proliferative activity among groups. In the control group, 14 days after balloon angioplasty, PCNA-positive cells in the neointimal area were less in the control group (n=5), compared with the TTM-treated group (2 weeks before/2 weeks after injury) (FIG. 10). Thus, TTM at a dose of 10 mg/kg, caused a significant reduction of the PCNA-positive cells in the (n=5; P<0.05).

S100a13, IL1, p40, PS Expression in Rat Balloon-Injured Vessel wall Treated with Copper Chelator TTM

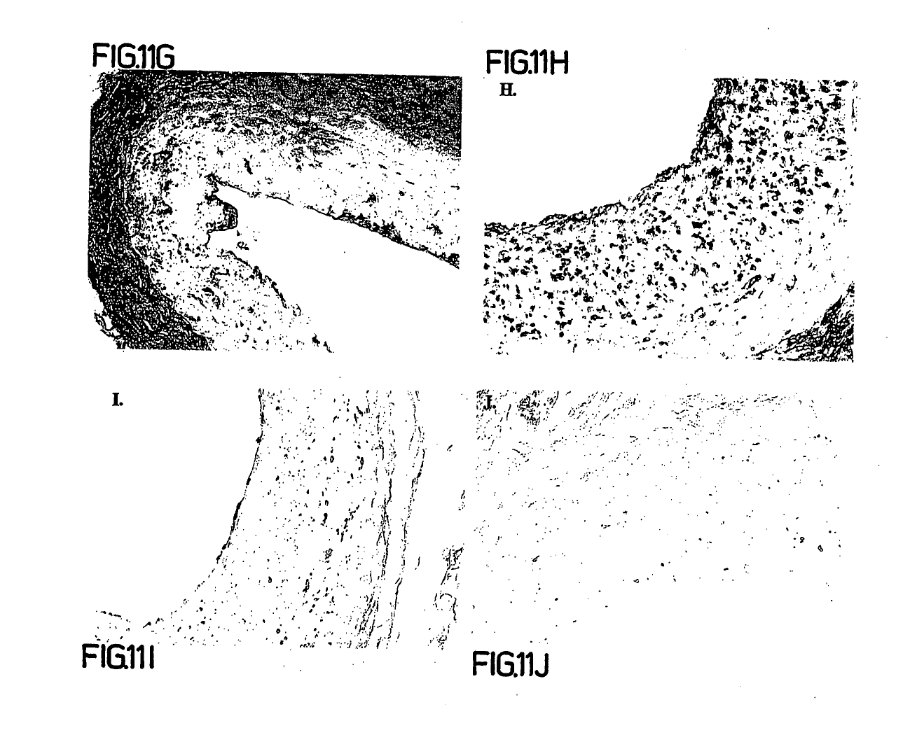

Four, seven and fourteen days after balloon injury, sections of the injured and uninjured arterial segments were analyzed for S100A13, IL1, p40 and PSby immunohistochemical analysis (n=5 each). In the balloon-injured arteries, there was time dependent diffuse expression of S100A13, IL1 and PS on the neointima and adventitia, whereas in the TTM-treated injured no positive staining was detected in the neointima 4 and 7 days after injury, and only a few cells were positively stained 14 days after the injury (FIG. 11).

The data disclosed herein demonstrate, for the first time, the role of copper chelation as a therapeutic tool for prevention of restenosis after balloon injury. The data disclosed demonstrate that copper chelation before and after balloon injury inhibits neointimal lesion formation by 53% in Sprague-Dawley rats due to strong antiproliferative and anti-inflammatory effects, likely due to the inhibition of FGF1 and IL1 from cells.

To expedite and sustain the end point of copper deficiency, ammonium tetrathiomolybdate, a potent and novel copper chelator, was utilized. TTM was developed originally for the treatment of Wilson's disease and was approved by the FDA as an orphan drug (Brewer et al., 1994, Arch. Neurol. 51:545-554; Brewer et al., 1991, Arch. Neurol. 48:42-47; Brewer et al., 1996, Arch. Neurol. 53:1017-1025). TTM forms a high-affinity tripartite complex with copper and albumin to chelate copper from the bloodstream (Ogra et al., 1998, J. Inorg. Biochem. 70:49-55; Ogra et al., 1996, Toxicology 106:75-83). TTM safely induces copper deficiency within 2-4 weeks in humans. Evidence from Phase I and preliminary results from Phase II clinical trials in patients with cancer demonstrate that humans can withstand significant copper deficiency induced by TTM with ceruloplasmin reduction to 20% of baseline for months and years (Brewer et al., 2000, Clin. Cancer Res. 6:1-10). The data disclosed herein demonstrate that copper deficiency in rats, with ceruloplasmin reduction to 0-20% of its baseline level, was induced and sustained by daily administration of 10 mg/kg TTM within 4 weeks without detectable effects upon visual inspection of the animals.

Neointimal formation after balloon injury is largely due to vascular smooth muscle cell proliferation, migration, differentiation, and activation with concomitant secretion of extracellular matrix. Theoretically, the SMC number in the neointima depends on cell migration, cell proliferation and apoptotic cell deaths. The data disclosed herein demonstrate that TTM significantly decreased the number of SMA-positive cells in the intima by 7 and 14 days after the injury, but no difference in the cell number was detected by day 4.

Moreover, histological analysis of treated vessels demonstrated intact vessel wall architecture and no alterations in overall all-morphology. However, in the balloon injured arteries, SMCs in the intima are first observed between days 3 and 4 after the injury (Frosen et al., 2001, Cardiovasc. Drugs Ther. 15:437-444). Since at day 4 SMCs should have undergone only limited SMC proliferation, intimal SMC number at day 4 post-injury is considered to represent the extent of SMC migration from the media, or more likely, the extent of myofibroblast migration from the adventitia (Frosen et al., 2001, Cardiovasc. Drugs Ther. 15:437-444; Shi et al., 1996, Circulation 94:1655-1664). Although SMC migration appears to be an important stage in post-injury neointimal formation in rat, its role in humans, where the balloon angioplasty is performed in vessels already narrowed by SMCs-rich atherosclerotic plaques, is rather minimal. In this respect, SMCs proliferation is much more important for the neointima development in humans.

Without wishing to be bound by any particular theory, since intimal thickening after balloon injury is a highly FGF-dependent process through FGF mitogenic activity (Lindner et al., 1995, Z. Kardiol. 84:137-144; Nabel et al., 1993, Nature 362:844-846), inhibition of FGF release may prevent neointimal development in response to injury. In addition, the role of FGF1 and FGF2 on neointimal development through their angiogenic activity should be considered as well (Edelman et al., 1992, J. Clin. Invest. 89:465473). Moreover, there is an increase in adventitial microvascular density early after balloon injury due to an active angiogenesis, which seems to occur beyond the first days but within the first week after the injury (Pels et al., 1999, Arterioscler. Thromb. Vasc. Biol. 19:229-238). This time frame is optimal for delivery of inflammatory cells and mesenchymal neointimal precursor cells for arterial repair. The inflammatory cell population triggers the differentiation of these cells and/or of the adventitial fibroblasts into myofibroblast, and their subsequent migration to the neointima (Pels et al., 1999, Arterioscler. Thromb. Vasc. Biol. 19:229-238; Scott et al., 1996, Circulation 93:2178-2187). As a rich source of FGF1, the inflammatory cell population augments the proliferative activity in the arterial wall after injury. Interestingly, the abundance of arterial wall microvessels starts to regress at the same time when the neointima mass accumulation sharply accelerates. Therefore, without wishing to be bound by any particular theory, the data disclosed herein suggest that adventitial angiogenesis early after balloon injury triggers the repair mechanisms in the vessel wall leading to neointimal formation.

Since copper metabolism appears to be fundamental for the stress-induced release of FGF1 into the extracellular compartment (Landriscina et al., 2001, J. Biol. Chem. 276:25549-25557), this mechanism may offer an explanation for the observed antiproliferative ability of TTM after balloon injury in the rat carotid artery. In accordance with this result, the data disclosed herein demonstrate that the number of cells expressing PCNA was significantly decreased in the neointima and media in the TTM-treated rats as compared to the controls. In addition, given the potent role of TTM as an anti-angiogenetic factor (Brewer et al., 2000, Clin. Cancer Res. 6:1-10; Frosen et al., 2001, Cardiovasc. Drugs Ther. 15:437-444; Shi et al., 1996, Circulation 94:1655-1664; Lindner et al., 1995, Z. Kardiol. 84:137-144; Nabel et al., 1993, Nature 362:844-846; Edelman et al., 1992, J. Clin. Invest. 89:465-473; Pels et al., 1999, Arterioscler. Thromb. Vasc. Biol. 19:229-238; Scott et al., 1996, Circulation 93:2178-2187; Cox et al., 2001, Laryngoscope 111:696-701), the decreased neointima formation in the TTM-treated rats 14 days after injury may be explained, in part and without wishing to be bound by any particular theory, by decreased adventitial angiogenesis due to copper chelation.

Inhibition of inflammation, as assessed by the extent of macrophage infiltration, was observed in the TTM treated rats as compared to the controls as demonstrated by the data disclosed herein. This finding demonstrates the role of copper chelation on macrophage attraction after balloon injury. The accumulation of macrophages in the neointima can result from “passive” retention of monocytes that would have passed through the arterial wall, or much more from active recruitment due to release of monocyte chemoattractants, including IL1, MCP1 (monocytes chemoattractant protein), IL-8, and the like (Okamoto et al., 2001, Circulation 104:2228-2235; Libby et al., 1992, Circulation 86:11147-11152). These chemokines have been found to be highly expressed in atherosclerotic lesions and after balloon injury, and facilitate SMC migration and proliferation (Okamoto et al., 2001, Circulation 104:2228-2235). It has been described previously that postangioplasty luminal loss in patients correlates with activation of circulating leukocytes. Furthermore, restenosis in patients that underwent atherectomy correlates with the percentage of macrophages in the retrieved tissue at the time of the atherectomy (Moreno et al., 1996, Circulation 94:3098-3102).

Recently, polymorphism of the gene for interleukin receptor antagonist, a protein that antagonizes IL1 for its receptor binding, was found to be associated with reduced restenosis (Kastrati et al., 2000, J. Am. Coll. Cardiol. 36:2168-2173; Francis et al., 2001, Heart 86:336-340). Furthermore, vascular injury in MAC-1-deficient mice is associated with reduced leukocyte accumulation and reduced neointima formation (Zou et al., 2000, Circ. Res. 86:434-440). The attenuated artery wall infiltration with macrophages in rats treated with TTM, observed in the data disclosed herein, is in accordance with the data disclosed previously elsewhere herein that the copper chelation diminishes the IL1 release in vitro (e.g., Example 1, supra). Indeed, the data disclosed herein demonstrate an almost complete inhibition in the IL1 positive staining 7 and 14 days after the balloon injury in TTM-treated rats, whereas the IL1 staining in the TTM-free rats was highly positive. In accordance with this finding is the significantly decreased presence of S00A13 in the TTM treated rats as compared to the controls 7 and 14 days after the injury.

IL1 and FGF1 release are both dependent on free copper ions, which participate in the formation of multiprotein release complex including S100A13 (Landriscina et al., 2001, J. Biol. Chem. 276:22544-22552). Copper chelation by TTM can inhibit both FGF1 and IL1 release with consequent inhibition of monocyte attraction and further decrease in S100A13, FGF and IL1 presence in the arterial wall.

It is well known that FGF prototypes do not contain a classical signal peptide sequence to direct their secretion into the extracellular compartment through the conventional exocytotic pathway mediated by endoplasmatic reticulum-Golgi apparatus. However, the release of the FGF prototypes have diverged, since only the FGF1 export pathway is inducible. The individual components that enable the FGF1 homodimer, as well as IL1, to utilize use the cytosolic face of a conventional intracellular vesicle to gain access to the intracellular surface of the plasma membrane, have been individually characterized as phosphatidylserine(pS)-binding protein. Indeed, IL1, FGF1, S100 gene family, p40 Syt 1, and annexin 2 are able to associate with pS under cell-free conditions. Phosphatidylserine is an acidic phospholipid, which is known to flip from the inner to the outer surface of plasma membranes in response to cellular stress. pS flipping is an important component of the intrinsic coagulation system and is widely used in its exaggerated form as evidence for cellular apoptotic behavior as a result of annexin 5 binding. If FGF1- or IL1-pS binding complex uses pS flipping for their export into the extracellular compartment in response to cellular stress, than their stress release should be tightly coupled to the appearance of pS in the outer leaflet of the plasma membrane. Indeed, the data disclosed herein demonstrate that the TTM treated rats did not show phosphatidylserine flipping whereas the controls did, demonstrating that release of these cytokines from a cell are associated with and/or mediated by pS flipping.

These experiments suggest that copper chelation effectively reduces neointima formation in vivo, and that copper chelation corresponds with a prominent antiproliferative and anti-inflammatory effect. The data disclosed herein also suggest that copper chelation can be a useful tool in the therapeutic management of vascular restenosis.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of inhibiting interleukin-1 alpha (IL-1α) release from a cell, said method comprising administering an effective amount of an IL-1α release inhibitor to said cell, thereby inhibiting IL-1α release from said cell.

2. The method of claim 1, wherein said release is stress-induced, and further wherein said IL-1α release inhibitor is selected from the group consisting of a copper chelator and a S100A13, or a fragment thereof.

3. The method of claim 3, wherein said S100A13 fragment is a S100A13ΔBR truncated protein.

4. The method of claim 4, wherein said copper chelator is tretrathiomolybdate (TTM).

5.-20. (canceled)
